SEQUENCE LISTING (84 sequences)

SEQ ID NO: 1 - protein, 133 aa, Homo sapiens [sequence omitted]

SEQ ID NO: 2 - protein, 133 aa, synthetic construct
  REGION 1..133: note = source = /note="Description of Artificial Sequence: Synthetic polypeptide"
QGQDRHMIRMRQLIDIVAQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 3 - protein, 133 aa, synthetic construct
  REGION 1..133: note = source = /note="Description of Artificial Sequence: Synthetic polypeptide"
QGQDRHMIRMRQLIDIVKQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 4 - protein, 133 aa, synthetic construct
  REGION 1..133: note = source = /note="Description of Artificial Sequence: Synthetic polypeptide"
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDS

SEQ ID NO: 5 - protein, 133 aa, synthetic construct
  REGION 1..133: note = source = /note="Description of Artificial Sequence: Synthetic polypeptide"
QGQDRHMIRMRQLIDIVDKLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDS

SEQ ID NO: 6 - protein, 133 aa, Homo sapiens [sequence omitted]

SEQ ID NO: 7 - protein, 113 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 8 - protein, 75 aa, Homo sapiens
ITCPPPMSVEHADIWVKSYSLYSRERYICNSGFKRKAGTSSLTECVLNKATNVAHWTTPSLKCIRDPALVHQRPA

SEQ ID NO: 9 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 10 - protein, 126 aa, Homo sapiens
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 11 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 12 - protein, 213 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 13 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 14 - protein, 219 aa, Homo sapiens
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 15 - protein, 230 aa, Homo sapiens
SVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWP

SEQ ID NO: 16 - protein, 500 aa, synthetic construct
  REGION 1..500: note = source = /note="Description of Artificial Sequence: Synthetic polypeptide"
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSVHMPLGFLGPRQARVVNGGGGGSGGGGSEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 17 - protein, 452 aa, synthetic construct
  REGION 1..452: note = source = /note="Description of Artificial Sequence: Synthetic polypeptide"
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSITCPPPMSVEHADIWVKSYSLYSRERYICNSGFKRKAGTSSLTECVLNKATNVAHWTTPSLKCIRGGGGSGGGSGGGGSAAGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 18 - protein, 240 aa, synthetic construct
  REGION 1..240: note = source = /note="Description of Artificial Sequence: Synthetic polypeptide"
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSGGGGSGGGGSGGGGSGIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEIS

SEQ ID NO: 19 - protein, 240 aa, synthetic construct
  REGION 1..240: note = source = /note="Description of Artificial Sequence: Synthetic polypeptide"
EIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 20 - protein, 247 aa, synthetic construct
  REGION 1..247: note = source = /note="Description of Artificial Sequence: Synthetic polypeptide"
QVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIK

SEQ ID NO: 21 - protein, 247 aa, synthetic construct
  REGION 1..247: note = source = /note="Description of Artificial Sequence: Synthetic polypeptide"
EIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 22 - protein, 248 aa, synthetic construct
  REGION 1..248: note = source = /note="Description of Artificial Sequence: Synthetic polypeptide"
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVDRYYNWNYFLGSFDYWGQGTLVTVSSGGGGSGGGGSGGGGSSYVLTQPPSVSVAPGKTARITCGGNNIRSKSVHWYQQKPGQAPVVVIYYDSDRPSGIPERISGSNSGNTATLTISRVEAGDEADYFCQVWDSSSDHHVFGGGTKLTVL

SEQ ID NO: 23 - protein, 248 aa, synthetic construct
  REGION 1..248: note = source = /note="Description of Artificial Sequence: Synthetic polypeptide"
SYVLTQPPSVSVAPGKTARITCGGNNIRSKSVHWYQQKPGQAPVVVIYYDSDRPSGIPERISGSNSGNTATLTISRVEAGDEADYFCQVWDSSSDHHVFGGGTKLTVLGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVDRYYNWNYFLGSFDYWGQGTLVTVSS

SEQ ID NO: 24 - protein, 588 aa, synthetic construct
  REGION 1..588: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 25 - protein, 675 aa, synthetic construct
  REGION 1..675: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGPLGVRGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 26 - protein, 692 aa, synthetic construct
  REGION 1..692: note = source = /note="Description of Artificial Sequence: Synthetic polypeptide"
MGVKVLFALICIAVAEAQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSAAGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 27 - protein, 20 aa, synthetic construct
  VARIANT 1..20: note = /note="This sequence may encompass 1-4 'Gly Gly Gly Gly Ser' repeating units"
  REGION 1..20: note = source = /note="Description of Artificial Sequence: Synthetic peptide"
GGGGSGGGGSGGGGSGGGGS

SEQ ID NO: 28 - protein, 5 aa, synthetic construct
  REGION 1..5: note = source = /note="Description of Artificial Sequence: Synthetic peptide"
GGGGS

SEQ ID NO: 29 - protein, 10 aa, synthetic construct
  REGION 1..10: note = source = /note="Description of Artificial Sequence: Synthetic peptide"
GGGGSGGGGS

SEQ ID NO: 30 - protein, 15 aa, synthetic construct
  REGION 1..15: note = source = /note="Description of Artificial Sequence: Synthetic peptide"
GGGGSGGGGSGGGGS

SEQ ID NO: 31 - protein, 22 aa, synthetic construct
  VARIANT 11..12: note = /replace="N"
  SITE 1..22: note = /note="Variant residues given in the sequence have no preference with respect to those in the annotations for variant positions"
  REGION 1..22: note = source = /note="Description of Artificial Sequence: Synthetic peptide"
GGGGSGGGGSAAGGGGSGGGGS

SEQ ID NO: 32 - protein, 27 aa, synthetic construct
  VARIANT 16..17: note = /replace="N"
  SITE 1..27: note = /note="Variant residues given in the sequence have no preference with respect to those in the annotations for variant positions"
  REGION 1..27: note = source = /note="Description of Artificial Sequence: Synthetic peptide"
GGGGSGGGGSGGGGSAAGGGGSGGGGS

SEQ ID NO: 33 - protein, 32 aa, synthetic construct
  VARIANT 16..17: note = /replace="N"
  SITE 1..32: note = /note="Variant residues given in the sequence have no preference with respect to those in the annotations for variant positions"
  REGION 1..32: note = source = /note="Description of Artificial Sequence: Synthetic polypeptide"
GGGGSGGGGSGGGGSAAGGGGSGGGGSGGGGS

SEQ ID NO: 34 - protein, 20 aa, synthetic construct
  VARIANT 10: note = /replace="A" or "N"
  VARIANT 15: note = /replace="N"
  SITE 1..20: note = /note="Variant residues given in the sequence have no preference with respect to those in the annotations for variant positions"
  REGION 1..20: note = source = /note="Description of Artificial Sequence: Synthetic peptide"
GGGGSGGGGSGGGGAGGGGS

SEQ ID NO: 35 - protein, 604 aa, synthetic construct
  VARIANT 440..454: note = /note="This region may encompass 0-3 'Gly Gly Gly Gly Ser' repeating units"
  VARIANT 455: note = /replace=" "
  VARIANT 456: note = /replace=" "
  VARIANT 457..471: note = /note="This region may encompass 0-3 'Gly Gly Gly Gly Ser' repeating units"
  VARIANT 474: note = /replace="A"
  VARIANT 506: note = /replace="N"
  VARIANT 513: note = /replace="G" or "S" or "T" or "Q" or "E" or "N" or "D" or "R" or "K"
  VARIANT 516: note = /replace="G" or "S" or "T" or "Q" or "E" or "N" or "D" or "R" or "K"
  VARIANT 543: note = /replace="G" or "S" or "T" or "Q" or "E" or "N" or "D" or "R" or "K"
  VARIANT 544: note = /replace="T"
  VARIANT 596: note = /replace="S"
  SITE 1..604: note = /note="Variant residues given in the sequence have no preference with respect to those in the annotations for variant positions"
  REGION 1..604: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLAGGGGSGGGGSGGGGSAAGGGGSGGGGSGGGGSAPNSSSTKKTQLQLEHLLLDLQMILNGINNYKNPKLTRMLTAKFAMPKKATELKHLQCLEEELKPLEEVLNAAQSKNFHLRPRDLISNINVIVLELKGSETTFMCEYADETATIVEFLNRWITFAQSIISTLT

SEQ ID NO: 36 - protein, 686 aa, synthetic construct
  VARIANT 441..455: note = /note="This region may encompass 0-3 'Gly Gly Gly Gly Ser' repeating units"
  VARIANT 456: note = /replace=" "
  VARIANT 457: note = /replace=" "
  VARIANT 458..472: note = /note="This region may encompass 0-3 'Gly Gly Gly Gly Ser' repeating units"
  SITE 1..686: note = /note="Variant residues given in the sequence have no preference with respect to those in the annotations for variant positions"
  REGION 1..686: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSAAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 37 - protein, 665 aa, synthetic construct
  REGION 1..665: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSITCPPPMSVEHADIWVKSYSLYSRERYICNSGFKRKAGTSSLTECVLNKATNVAHWTTPSLKCIRGGGGSGGGSGGGGSAAGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 38 - protein, 712 aa, synthetic construct
  VARIANT 441..455: note = /note="This region may encompass 0-3 'Gly Gly Gly Gly Ser' repeating units"
  VARIANT 456: note = /replace=" "
  VARIANT 457: note = /replace=" "
  VARIANT 458..472: note = /note="This region may encompass 0-3 'Gly Gly Gly Gly Ser' repeating units"
  SITE 1..712: note = /note="Variant residues given in the sequence have no preference with respect to those in the annotations for variant positions"
  REGION 1..712: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSAAGGGGSGGGGSGGGGSEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 39 - protein, 605 aa, synthetic construct
  REGION 1..605: note = source = /note="Description of Artificial Sequence: Synthetic polypeptide"
MGVKVLFALICIAVAEAQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 40 - protein, 691 aa, synthetic construct
  REGION 1..691: note = source = /note="Description of Artificial Sequence: Synthetic polypeptide"
MGVKVLFALICIAVAEAQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 41 - protein, 726 aa, synthetic construct
  REGION 1..726: note = source = /note="Description of Artificial Sequence: Synthetic polypeptide"
MGVKVLFALICIAVAEAQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSAAGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 42 - protein, 588 aa, synthetic construct
  REGION 1..588: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 43 - protein, 675 aa, synthetic construct
  REGION 1..675: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSAAGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 44 - protein, 214 aa, synthetic construct
  REGION 1..214: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 214 aa, synthetic construct
  REGION 1..214: note = source = /note="Description of Artificial Sequence: Synthetic polypeptide"
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 448 aa, synthetic construct
  REGION 1..448: note = source = /note="Description of Artificial Sequence: Synthetic polypeptide"
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 440 aa, synthetic construct
  REGION 1..440: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 218 aa, synthetic construct
  REGION 1..218: note = source = /note="Description of Artificial Sequence: Synthetic polypeptide"
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 447 aa, synthetic construct
  REGION 1..447: note = source = /note="Description of Artificial Sequence: Synthetic polypeptide"
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 215 aa, synthetic construct
  REGION 1..215: note = source = /note="Description of Artificial Sequence: Synthetic polypeptide"
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 613 aa, synthetic construct
  VARIANT 449..463: note = /note="This region may encompass 0-3 'Gly Gly Gly Gly Ser' repeating units"
  VARIANT 464: note = /replace=" "
  VARIANT 465: note = /replace=" "
  VARIANT 466..480: note = /note="This region may encompass 0-3 'Gly Gly Gly Gly Ser' repeating units"
  VARIANT 483: note = /replace="A"
  VARIANT 515: note = /replace="N"
  VARIANT 522: note = /replace="G" or "S" or "T" or "Q" or "E" or "N" or "D" or "R" or "K"
  VARIANT 525: note = /replace="G" or "S" or "T" or "Q" or "E" or "N" or "D" or "R" or "K"
  VARIANT 552: note = /replace="G" or "S" or "T" or "Q" or "E" or "N" or "D" or "R" or "K"
  VARIANT 553: note = /replace="T"
  VARIANT 605: note = /replace="S"
  SITE 1..613: note = /note="Variant residues given in the sequence have no preference with respect to those in the annotations for variant positions"
  REGION 1..613: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAAGGGGSGGGGSGGGGSAPNSSSTKKTQLQLEHLLLDLQMILNGINNYKNPKLTRMLTAKFAMPKKATELKHLQCLEEELKPLEEVLNAAQSKNFHLRPRDLISNINVIVLELKGSETTFMCEYADETATIVEFLNRWITFAQSIISTLT

SEQ ID NO: 52 - protein, 673 aa, synthetic construct
  REGION 1..673: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSITCPPPMSVEHADIWVKSYSLYSRERYICNSGFKRKAGTSSLTECVLNKATNVAHWTTPSLKCIRGGGGSGGGSGGGGSAAGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 53 - protein, 613 aa, synthetic construct
  VARIANT 449..463: note = /note="This region may encompass 0-3 'Gly Gly Gly Gly Ser' repeating units"
  VARIANT 464: note = /replace=" "
  VARIANT 465: note = /replace=" "
  VARIANT 466..480: note = /note="This region may encompass 0-3 'Gly Gly Gly Gly Ser' repeating units"
  SITE 1..613: note = /note="Variant residues given in the sequence have no preference with respect to those in the annotations for variant positions"
  REGION 1..613: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAAGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 54 - protein, 694 aa, synthetic construct
  VARIANT 449..463: note = /note="This region may encompass 0-3 'Gly Gly Gly Gly Ser' repeating units"
  VARIANT 464: note = /replace=" "
  VARIANT 465: note = /replace=" "
  VARIANT 466..480: note = /note="This region may encompass 0-3 'Gly Gly Gly Gly Ser' repeating units"
  SITE 1..694: note = /note="Variant residues given in the sequence have no preference with respect to those in the annotations for variant positions"
  REGION 1..694: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAAGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 55 - protein, 728 aa, synthetic construct
  VARIANT 449..463: note = /note="This region may encompass 0-3 'Gly Gly Gly Gly Ser' repeating units"
  VARIANT 464: note = /replace=" "
  VARIANT 465: note = /replace=" "
  VARIANT 466..480: note = /note="This region may encompass 0-3 'Gly Gly Gly Gly Ser' repeating units"
  SITE 1..728: note = /note="Variant residues given in the sequence have no preference with respect to those in the annotations for variant positions"
  REGION 1..728: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAAGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVDRYYNWNYFLGSFDYWGQGTLVTVSSGGGGSGGGGSGGGGSSYVLTQPPSVSVAPGKTARITCGGNNIRSKSVHWYQQKPGQAPVVVIYYDSDRPSGIPERISGSNSGNTATLTISRVEAGDEADYFCQVWDSSSDHHVFGGGTKLTVL

SEQ ID NO: 56 - protein, 728 aa, synthetic construct
  VARIANT 449..463: note = /note="This region may encompass 0-3 'Gly Gly Gly Gly Ser' repeating units"
  VARIANT 464: note = /replace=" "
  VARIANT 465: note = /replace=" "
  VARIANT 466..480: note = /note="This region may encompass 0-3 'Gly Gly Gly Gly Ser' repeating units"
  SITE 1..728: note = /note="Variant residues given in the sequence have no preference with respect to those in the annotations for variant positions"
  REGION 1..728: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAAGGGGSGGGGSGGGGSSYVLTQPPSVSVAPGKTARITCGGNNIRSKSVHWYQQKPGQAPVVVIYYDSDRPSGIPERISGSNSGNTATLTISRVEAGDEADYFCQVWDSSSDHHVFGGGTKLTVLGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVDRYYNWNYFLGSFDYWGQGTLVTVSS

SEQ ID NO: 57 - protein, 720 aa, synthetic construct
  VARIANT 449..463: note = /note="This region may encompass 0-3 'Gly Gly Gly Gly Ser' repeating units"
  VARIANT 464: note = /replace=" "
  VARIANT 465: note = /replace=" "
  VARIANT 466..480: note = /note="This region may encompass 0-3 'Gly Gly Gly Gly Ser' repeating units"
  SITE 1..720: note = /note="Variant residues given in the sequence have no preference with respect to those in the annotations for variant positions"
  REGION 1..720: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAAGGGGSGGGGSGGGGSEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 58 - protein, 683 aa, synthetic construct
  REGION 1..683: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSAAGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 59 - protein, 717 aa, synthetic construct
  REGION 1..717: note = source = /note="Description of Artificial Sequence: Synthetic polypeptide"
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSAAGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 60 - protein, 124 aa, synthetic construct
  REGION 1..124: note = source = /note="Description of Artificial Sequence: Synthetic polypeptide"
EVQLQQSGAELVRPGASVKLSCTASGFNIKDDYLHWVRQRPEQGLEWIGRIDPANGNTKYAPKFQDKATITADTSSNTAYLQLSILTSEDTAVYYCAARFGYFYGSSFYAVAYWGQGTSVTVSS

SEQ ID NO: 61 - protein, 106 aa, synthetic construct
  REGION 1..106: note = source = /note="Description of Artificial Sequence: Synthetic polypeptide"
QIVLTQSPAIMSASPGEKVTMTCSASSSVSYMHWYQQKSGTSPKRWIYDTSKLASGVPARFSGSGSGTSYSLTISSMEAEDAATYYCQQWSSNPYTFGGGTKLEIK

SEQ ID NO: 62 - protein, 133 aa, synthetic construct
  VARIANT 3: note = /replace="A"
  VARIANT 35: note = /replace="N"
  VARIANT 42: note = /replace="G" or "S" or "T" or "Q" or "E" or "N" or "D" or "R" or "K"
  VARIANT 45: note = /replace="G" or "S" or "T" or "Q" or "E" or "N" or "D" or "R" or "K"
  VARIANT 72: note = /replace="G" or "S" or "T" or "Q" or "E" or "N" or "D" or "R" or "K"
  VARIANT 73: note = /replace="T"
  VARIANT 125: note = /replace="A" or "S"
  SITE 1..133: note = /note="Variant residues given in the sequence have no preference with respect to those in the annotations for variant positions"
  REGION 1..133: note = source = /note="Description of Artificial Sequence: Synthetic polypeptide"
APNSSSTKKTQLQLEHLLLDLQMILNGINNYKNPKLTRMLTAKFAMPKKATELKHLQCLEEELKPLEEVLNAAQSKNFHLRPRDLISNINVIVLELKGSETTFMCEYADETATIVEFLNRWITFCQSIISTLT

SEQ ID NO: 63 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDGSGVSPRRKLISVDSRSVSLLPLEFRKDSSYELQVRAGPMPGSSYQGTWSEWSDPVIFQTQSEELKE

SEQ ID NO: 64 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDNASVSPRRKLISVDSRSVSLLPLEFRKDSSYELQVRAGPMPGSSYQGTWSEWSDPVIFQTQSEELKE

SEQ ID NO: 65 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDNGSVSPRRKLISVDSRSVSLLPLEFRKDSSYELQVRAGPMPGSSYQGTWSEWSDPVIFQTQSEELKE

SEQ ID NO: 66 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLNRSAHNATHATYTCHMDVFHFMADDIFSVNITDQSGNYSQECGSFLLAESIKPAPPFNVTVTFSGQYNISWRSDYEDPAFYMLKGKLQYELQYRNRGDNASVSPRRKLISVDSRSVSLLPLEFRKDSSYELQVRAGPMPGSSYQGTWSEWSDPVIFQTQSEELKE

SEQ ID NO: 67 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPSAVSPRRKLISVDSRSVSLLPLEFRKDSSYELQVRAGPMPGSSYQGTWSEWSDPVIFQTQSEELKE

SEQ ID NO: 68 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPSGVSPRRKLISVDSRSVSLLPLEFRKDSSYELQVRAGPMPGSSYQGTWSEWSDPVIFQTQSEELKE

SEQ ID NO: 69 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSAYEDPAFYMLKGKLQYELQYRNRGDGSGVSPRRKLISVDSRSVSLLPLEFRKDSSYELQVRAGPMPGSSYQGTWSEWSDPVIFQTQSEELKE

SEQ ID NO: 70 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSIYEDPAFYMLKGKLQYELQYRNRGDGSGVSPRRKLISVDSRSVSLLPLEFRKDSSYELQVRAGPMPGSSYQGTWSEWSDPVIFQTQSEELKE

SEQ ID NO: 71 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSWYEDPAFYMLKGKLQYELQYRNRGDGSGVSPRRKLISVDSRSVSLLPLEFRKDSSYELQVRAGPMPGSSYQGTWSEWSDPVIFQTQSEELKE

SEQ ID NO: 72 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSFYEDPAFYMLKGKLQYELQYRNRGDGSGVSPRRKLISVDSRSVSLLPLEFRKDSSYELQVRAGPMPGSSYQGTWSEWSDPVIFQTQSEELKE

SEQ ID NO: 73 - protein, 213 aa, synthetic construct
  REGION 1..213: note = source = /note="Description of Artificial Sequence: Synthetic polypeptide"
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSYYEDPAFYMLKGKLQYELQYRNRGDGSGVSPRRKLISVDSRSVSLLPLEFRKDSSYELQVRAGPMPGSSYQGTWSEWSDPVIFQTQSEELKE

SEQ ID NO: 74 - protein, 674 aa, synthetic construct
  REGION 1..674: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGPLGVRGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 75 - protein, 706 aa, synthetic construct
  REGION 1..706: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISGGGGSGGGGSGGGGSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 76 - protein, 5 aa, synthetic construct
  REGION 1..5: note = source = /note="Description of Artificial Sequence: Synthetic peptide"
TYWIG

SEQ ID NO: 77 - protein, 17 aa, synthetic construct
  REGION 1..17: note = source = /note="Description of Artificial Sequence: Synthetic peptide"
IIYPGDSDTRYSPSFQG

SEQ ID NO: 78 - protein, 8 aa, synthetic construct
  REGION 1..8: note = source = /note="Description of Artificial Sequence: Synthetic peptide"
GNWNCFDY

SEQ ID NO: 79 - protein, 12 aa, synthetic construct
  REGION 1..12: note = source = /note="Description of Artificial Sequence: Synthetic peptide"
RASQSVSSSYLA

SEQ ID NO: 80 - protein, 7 aa, synthetic construct
  REGION 1..7: note = source = /note="Description of Artificial Sequence: Synthetic peptide"
GASRRAT

SEQ ID NO: 81 - protein, 8 aa, synthetic construct
  REGION 1..8: note = source = /note="Description of Artificial Sequence: Synthetic peptide"
QRYGSSHT

SEQ ID NO: 82 - protein, 8 aa, synthetic construct
  REGION 1..8: note = source = /note="Description of Artificial Sequence: Synthetic peptide"
GNWNSFDY

SEQ ID NO: 83 - protein, 118 aa, synthetic construct
  REGION 1..118: note = source = /note="Description of Artificial Sequence: Synthetic polypeptide"
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 84 - protein, 119 aa, synthetic construct
  REGION 1..119: note = source = /note="Description of Artificial Sequence: Synthetic polypeptide"
EIVLTQSPGTLSLSPGREATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFP

(19) United States
(12) Patent Application Publication
(10) Pub. No.: US 2026/0250354 A1
(43) Pub. Date: Aug. 27, 2026

(54) MASKED CYTOKINES AND METHODS OF USE THEREOF

(71) Applicant: ASKGENE PHARMA, INC., Camarillo, CA (US)

(72) Inventors: Yuefeng LU, Moorpark, CA (US); Chunxiao YU, Santa Barbara, CA (US); Jian-Feng LU, Oak Park, CA (US); Liqin LIU, Woodland Hills, CA (US)

(73) Assignee: ASKGENE PHARMA, INC., Camarillo, CA (US)

(21) Appl. No.: 19/548,829

(22) Filed: Feb. 24, 2026

Related U.S. Application Data
(62) Division of application No. 17/787,741, filed on Jan. 11, 2021, now Pat. No. 12,600,762.
(60) Provisional application No. 62/959,973, filed on Jan. 11, 2020.
(60) Provisional application No. 63/027,138, filed on May 19, 2020.
(60) Provisional application No. 63/029,473, filed on May 23, 2020.
(60) Provisional application No. 63/126,393, filed on Dec. 16, 2020.

(51) Int. Cl. C07K 14/715; A61K 38/00; C07K 14/54; C07K 14/55; C07K 16/24; C07K 16/28

(52) U.S. Cl. CPC C07K 14/7155; C07K 14/54; C07K 14/5418; C07K 14/5443; C07K 14/55; C07K 16/244; C07K 16/2818; A61K 38/00; C07K 2317/622; C07K 2319/00

(57) ABSTRACT

Provided herein are cytokine prodrugs and methods of making and using thereof in stimulating the immune system, or treating cancer, an autoimmune disease, or an infectious disease.

### CROSS REFERENCE TO RELATED APPLICATIONS

This application is a divisional of U.S. patent application Ser. No. 17/787,741, filed Jun. 21, 2022, which is a national stage application under 35 U.S.C. § 371 of International Patent Application No. PCT/US2021/013007, filed Jan. 11, 2021, which claims priority from U.S. Provisional Application Nos. 62/959,973, filed Jan. 11, 2020; 63/027,138, filed May 19, 2020; 63/029,473, filed May 23, 2020; and 63/126,393, filed Dec. 16, 2020, the disclosures of which are incorporated herein by reference in their entirety.

### SEQUENCE LISTING

The instant application contains a Sequence Listing which has been submitted electronically in XML format and is hereby incorporated by reference in its entirety. Said XML copy, created on Feb. 24, 2026, is named 025471.D1008.xml and is 164,482 bytes in size.

### BACKGROUND OF THE INVENTION

Interleukin (IL)-2, IL-4, IL-7, IL-9, IL-15, and IL-21 play important roles in the proliferation, differentiation, and survival of immune cells. The receptors for these cytokines share a common γ chain (γC), also known as CD132. Thus, these cytokines are also called common γC family cytokines.

IL-2 plays a central role in lymphocyte generation, survival, and homeostasis. It has 133 amino acids and consists of four antiparallel, amphipathic alpha-helices that form a quaternary structure essential for its function (Smith, Science (1988) 240:1169-76; Bazan, Science (1992) 257:410-13). IL-2 exerts its activities by binding to IL-2 receptors (IL-2R), which consist of up to three individual subunits. Association of the α (CD25 or Tac antigen), β (CD122), and γC subunits results in a trimeric, high-affinity receptor for IL-2 (K_{D} ~0.01 nM). Dimeric IL-2 receptor consisting of the β and γ subunits is termed intermediate-affinity IL-2R (K_{D} ~1 nM). The a subunit alone forms the monomeric low affinity IL-2 receptor (K_{D} ~10 nM). See, e.g., Kim et al., Cytokine Growth Factor Rev. (2006) 17:349-66). Although the dimeric intermediate-affinity IL-2 receptor binds IL-2 with approximately 100-fold lower affinity than the trimeric high-affinity receptor, both the dimeric and trimeric IL-2 receptors can transmit signal upon IL-2 binding (Minami et al., Annu Rev Immunol. (1993) 11:245-68).

IL-15 is a cytokine with structural similarities to IL-2. IL-15 is secreted by mononuclear phagocytes and other immune cells following viral infection. IL-15 induces proliferation of natural killer (NK) and other cells of the immune system and is involved in the killing of virally infected cells and cancer cells. Like IL-2, IL-15 binds to the IL-2 receptor (IL-2R) β/γ complex, the intermediate affinity receptor, with a K_{D} of about 1 nM (Giri et al., EMBO J. (1994) 13:2822-30). IL-15 binds to IL-15 receptor (IL-15R) α with a much higher affinity (K_{D} ~0.05 nM). IL-15Rα can associate with the IL-2Rβ/γ complex to form an IL-15-specific, functional high-affinity (αβγ) receptor (Minami et al., Annu. Rev. Immunol. (1993) 11:245-67; Giri et al., J Leukoc Biol. (1995) 5745:763-6; and Lehours et al., Eur Cytokine Netw. (2000) 11:207-15).

IL-21 is produced by activated CD4+ T cells, T-follicular helper cells, and natural killer T (NKT) cells (Spolski and Leonard, Ann Rev Immunol. (2008) 26:57008). IL-21 has been shown to exert pleiotropic effects on the proliferation, differentiation, and cytotoxicity of various classes of lymphoid cells. More recently, IL-21 has been further shown to play a crucial role in the differentiation of CD4⁺ T cells into T-helper 17 (TH₁₇) cells, a subset of T cells associated with development of inflammatory conditions and autoimmune diseases (Korn et al., Nature (2007) 448(7152):484-87; Nurieva et al., Nature (2007) 448(7152):480-83). The receptor complex of IL-21 is composed of the private chain IL-21Rα and the common chain γC (or Ry). Human IL-21 binds to IL-21Rα with a very high affinity (K_{D} ~70 pM; Zhang et al., Biochem Biophys Res Commun. (2003) 300(2):291-6), while binding to IL-21Rγ with a relatively low affinity (K_{D} ~160 M).

The above cytokines, their muteins, and fusion proteins have been investigated for their potential as therapeutics, with recombinant IL-2 being the first cytokine approved for cancer therapy. However, these cytokine drugs and drug candidates have significant side effects. In addition, their in vivo half-lives are often short, even when presented as antibody-cytokine fusion molecules, potentially due to the “PK sink” formed by the receptors of the cytokines on the immune cells.

Thus, there remains a need to develop cytokine-based cancer therapeutics that are more tumor site-selective and have improved PK and efficacy, while causing fewer side effects.

### SUMMARY OF THE INVENTION

The present disclosure provides a prodrug comprising a cytokine moiety, a masking moiety, and a carrier moiety, wherein the masking moiety binds to the cytokine moiety and inhibits a biological activity of the cytokine; the carrier moiety comprises an antigen-binding moiety that binds to an antigen expressed on the surface of a target cell; and the masking moiety is linked to the carrier moiety without a cleavable peptide linker (e.g., linked indirectly to the carrier moiety, through a non-cleavable peptide linker; or directly, without a peptide linker).

In some embodiments, the prodrug is activated by cells that express on the cell surface both the antigen and a receptor for the cytokine moiety. In some embodiments, the biological activity of the prodrug is increased by at least 2, 5, or 10 folds at a disease site comprising the cells as compared to a site not comprising the cells. In particular embodiments, the receptor for the cytokine moiety comprises two or more subunits.

In some embodiments, the antigen is selected from PD-1, PD-L1, CTLA-4, TIGIT, TIM-3, LAG-3, CD25, CD16a, CD16b, NKG2D, NKP44, NKP30, CD19, CD20, CD30, CD38, BCMA, and signal regulatory protein alpha (SIRP alpha, designated CD172a).

In some embodiments, the cytokine moiety comprises an IL-2 agonist polypeptide, an IL-7 agonist polypeptide, an IL-9 agonist polypeptide, an IL-15 agonist polypeptide, an IL-21 agonist polypeptide, an IL-1α agonist polypeptide, an IL-1β agonist polypeptide, an IL-4 agonist polypeptide, an IL-5 agonist polypeptide, an IL-6 agonist polypeptide, an IL-8 agonist polypeptide, an IL-10 agonist polypeptide, an IL-12 agonist polypeptide, an IL-17 agonist polypeptide, an IL-18 agonist polypeptide, an IL-22 agonist polypeptide, an IL-23 agonist polypeptide, an IL-31 agonist polypeptide, an IL-33 agonist polypeptide, an IL-36 agonist polypeptide, an interferon-alpha agonist polypeptide, an interferon gamma agonist polypeptide, a 4-1BB ligand, an OX-40 ligand, or a CD-40 ligand.

In some embodiments, the cytokine moiety comprises an IL-2 agonist polypeptide or an IL-15 agonist polypeptide and the masking moiety comprises an ECD of IL-2 receptor β (IL-2Rβ ECD) or a functional fragment thereof. In additional embodiments, the cytokine moiety comprises an IL-21 agonist polypeptide and the masking moiety comprises a Fab, a single chain Fv (scFv), or a single domain antibody against IL-21. In other embodiments, the cytokine moiety comprise an IL-2 agonist polypeptide and the masking moiety comprises a Fab, an scFv, or a single domain antibody against IL-2. In additional embodiments, the cytokine moiety comprises an IL-15 agonist polypeptide, and the masking moiety comprises a Fab, an scFv, or a single domain antibody against IL-15. In other embodiments, the cytokine moiety comprises an IL-15 agonist polypeptide, and the masking moiety comprises a sushi domain of IL-15 receptor α (IL-15Rα sushi domain).

In some embodiments, the cytokine moiety comprises an IL-21 agonist polypeptide comprising SEQ ID NO: 1, or an amino acid sequence at least 90% identical thereto. In other embodiments, the cytokine moiety comprises an IL-2 agonist polypeptide comprising SEQ ID NO: 6 or 62, or an amino acid sequence at least 95% identical thereto. In other embodiments, the cytokine moiety comprises an IL-15 agonist polypeptide comprising SEQ ID NO: 7, or an amino acid sequence at least 95% identical thereto.

In some embodiments, the masking moiety comprises an extracellular domain (ECD) of a receptor of the cytokine. In particular embodiments, the cytokine moiety comprises an IL-7 agonist polypeptide and the masking moiety comprises an ECD of IL-7 receptor α (IL-7Rα ECD) or a functional analog thereof. In particular embodiments, the cytokine moiety is an IL-21 agonist polypeptide and the masking moiety comprises an ECD of IL-21 receptor α (IL-21Rα ECD) or a functional analog thereof. In particular embodiments, the masking moiety comprises an IL-21Rα ECD or a functional analog thereof comprising an amino acid sequence selected from SEQ ID NO: 12, 13, and 63-73, or at least 90% (e.g., at least 95%) identical thereto. In some embodiments, the masking moiety comprises an IL-2Rβ ECD or a functional analog thereof comprising SEQ ID NO: 11, or an amino acid sequence at least 95% identical thereto.

In some embodiments, the masking moiety comprises an scFv that binds the cytokine moiety and inhibits or interferes with the interaction between the cytokine moiety and its receptors. In particular embodiments, the masking moiety is an scFv that binds to human IL-21 and comprises an amino acid sequence of SEQ ID NO: 20 or 21. In particular embodiments, the masking moiety comprises an scFv that binds to IL-2 and inhibits or interferes with the interaction between IL-2 and IL-2Rα, the interaction between IL-2 and IL-2Rβ, and/or the interaction between IL-2 and IL-2Rγ, optionally wherein the scFv comprises the heavy chain and light chain CDRs of antibody 4E12B2D10. In some embodiments, the scFv comprises the heavy chain and light chain of antibody 4E12B2D10. In particular embodiments, the heavy chain and light chain amino acid sequences of 4E12B2D10 comprise SEQ ID NO: 60 and 61, respectively. In some embodiments, the masking moiety is an scFv that binds to IL-2 and comprises an amino acid sequence of SEQ ID NO: 22 or 23, or at least 95% identical as that of SEQ ID NO: 22 or 23. In particular embodiments, the masking moiety comprises an scFv that binds to IL-15 and inhibits or interferes with the interaction between IL-15 and IL-2Rβ, and/or the interaction between IL-2 and IL-2Rγ, optionally wherein the scFv comprises the heavy chain and light chain CDRs of anti-IL-15 antibody 146B7, 146H5, or 404E4, or optionally wherein the scFv comprises the heavy chain and light chain of anti-IL-15 antibody 146B7, 146H5, or 404E4, or optionally wherein the scFv comprises SEQ ID NO: 18 or 19. In some embodiments, an scFv or Fab IL-15 antagonist comprises heavy chain CDR1-3 of an anti-IL-15 antibody selected from 146B7, 146H5, and 404E4; and the light chain CDR1-3 of anti-IL-15 antibody selected from 146B7, 146H5, and 404E4 all of which are described in described in WO2003/017935A2.

In particular embodiments, the masking moiety comprises an scFv that binds to IL-21 and inhibits or interferes with the interaction between IL-21 and IL-21Rα, and/or the interaction between IL-21 and IL-2Rγ. In particular embodiments, the masking moiety comprises an scFv that binds to a cytokine selected from IL-2, IL-7, IL-9, IL-15, or IL-21 and inhibits or interferes with the interaction between the cytokine and IL-2Rγ.

In some embodiments, the prodrug has a half-life in a non-human primate or a human that is at least 20 times, at least 50 times, at least 100 times, at least 150 times, or at least 200 times longer than that of the corresponding wild type cytokine.

In particular embodiments, the prodrug comprises two light chains with an amino acid sequence of SEQ ID NO: 44, and two heavy chain polypeptide chains whose amino acid sequences respectively comprise SEQ ID NOs: 24 and 25; SEQ ID NOs: 35 and 36; SEQ ID NOs: 37 and 36; SEQ ID NOs: 37 and 38; SEQ ID NOs: 39 and 41; or SEQ ID NOs: 42 and 43.

In particular embodiments, the prodrug comprises two light chains with an amino acid sequence of SEQ ID NO: 50, and two heavy chain polypeptide chains whose amino acid sequences respectively comprise SEQ ID NOs: 51 and 54; SEQ ID NOs: 51 and 55; SEQ ID NOs: 51 and 56; SEQ ID NOs: 52 and 54; SEQ ID NOs: 53 and 58; SEQ ID NOs: 53 and 59; or SEQ ID NOs: 52 and 57.

In some embodiments, the present disclosure provides a pharmaceutical composition comprising the prodrug of the present disclosure and a pharmaceutically acceptable excipient.

In some aspects, the present disclosure provides a polynucleotide or polynucleotides encoding the present prodrugs, expression vectors comprising the polynucleotides, and host cells (e.g., mammalian host cells such as CHO, NS0 cells, and 293T cells) comprising the expression vectors. The present disclosure also provides methods of making the present prodrugs, comprising culturing the host cells under conditions that allow expression of prodrugs and isolating (also purifying) the prodrugs.

An Fc-based IL-21 prodrug molecule was not able to bind to cells such as Mino cells which expresses IL-21 receptors (FIG. 2), confirming that the cytokine moieties were masked. It was further shown that the masked molecules with PD-1 antibody as the carrier had minimum activities with the NK92 cells which do not express PD-1 (FIG. 3). It was a surprise to the inventors that the PD-1 antibody-based IL-21 prodrug molecules had meaningful activities with the Mino cells prior to activation (FIGS. 4A and 4B). Similar observations were made with an IL-2 prodrug (FIGS. 6A and 6B) and an IL-15 prodrug (FIGS. 5A-C). While not wishing to be bound by any theory, it is hypothesized that cis-binding of the PD-1 antibody to the antigen and the cytokine moiety to the cytokine receptors respectively, wherein both the antigen and the cytokine receptors are expressed on the same cell, led to the undoing of the masking effect of the masking moiety. It is therefore proposed that the prodrugs with the targeting moiety provides a significant selectivity to the cells expressing the antigens targeted by the carried moiety over the ones without the antigens. Surprisingly, it is feasible to construct prodrugs without the need of protease cleavage or removal of the masking moiety.

The present disclosure provides prodrugs which can be selectively activated at a disease site without the need of protease cleavage or removal of the masking moiety. In one aspect, the present disclosure provides novel cytokines prodrug comprising a cytokine moiety (a cytokine agonist polypeptide), a masking moiety, and a carrier moiety, wherein the masking moiety binds to the cytokine agonist polypeptide and inhibits an intended biological activity of the cytokine; the carrier moiety comprises an antigen binding moiety; the masking moiety is linked directly or indirectly to the carrier moiety through a non-cleavable peptide linker or without a peptide linker; and wherein the activity of the prodrug is higher in stimulating a cell which expresses both the antigen targeted by the carrier moiety and the receptor(s) of the cytokine than its activity in stimulating a cell which expresses the receptor(s) of the cytokine but not the antigen targeted by the carrier moiety.

In one aspect, the present disclosure provides novel cytokines prodrug comprising a cytokine moiety (a cytokine agonist polypeptide), a masking moiety, and a carrier moiety, wherein the masking moiety binds to the cytokine agonist polypeptide and inhibits an intended biological activity of the cytokine; the carrier moiety comprises an antigen binding moiety; the masking moiety is linked directly or indirectly to the carrier moiety through a non-cleavable peptide linker or without a peptide linker; and wherein the prodrug is activated at a disease site where there are cells expressing both the antigen targeted by the carrier moiety and the receptor(s) of the cytokine, or a biological activity of the prodrug is increased by at least 2 folds, at least 5 folds, or at least 10 folds at a disease site where there are cells expressing both the antigen targeted by the carrier moiety and the receptor(s) of the cytokine.

In some embodiments, the prodrug does not contain any cleavable peptide linker.

In some embodiments, the carrier moiety comprises an antigen-binding moiety, wherein the antigen is expressed on an immune cell. In some embodiments, the carrier moiety comprises an antigen-binding moiety, wherein the antigen is selected from PD-1, PD-L1, CTLA-4, TIGIT, TIM-3, LAG-3, CD25, CD16a, and CD16b.

In some embodiments, the cytokine moiety comprises a cytokine selected from an IL-2 agonist polypeptide, an IL-7 agonist polypeptide, an IL-9 agonist polypeptide, an IL-15 agonist polypeptide, and an IL-21 agonist polypeptide. In some embodiments, the cytokine is selected from an IL-1α agonist polypeptide, an IL-1β agonist polypeptide, an IL-4 agonist polypeptide, an IL-5 agonist polypeptide, an IL-6 agonist polypeptide, an IL-8 agonist polypeptide, an IL-10 agonist polypeptide, an IL-12 agonist polypeptide, an IL-15 agonist polypeptide, an IL-17 agonist polypeptide, an IL-18 agonist polypeptide, an IL-22 agonist polypeptide, an IL-23 agonist polypeptide, an IL-31 agonist polypeptide, an IL-33 agonist polypeptide, an IL-36 agonist polypeptide, an Interferon-alpha agonist polypeptide, interferon gamma, 4-1BB ligand, OX-40 ligand, CD-40 ligand.

In some embodiments, the masking moiety is an extracellular domain (ECD) of a receptor of the cytokine. In some embodiments, the masking moiety comprises an antibody against the cytokine or a binding fragment of the antibody.

In some embodiments, the cytokine is an IL-21 agonist polypeptide; and wherein the masking moiety is the extracellular domain of IL-21 receptor α (IL-21Rα ECD) or a functional analog thereof. In some embodiments, the cytokine is an IL-2 agonist polypeptide or an IL-15 agonist polypeptide; and wherein the masking moiety is the extracellular domain of IL-2 receptor β (IL-2Rβ ECD). In some embodiments, the cytokine is an IL-21 agonist polypeptide; and wherein the masking moiety is a Fab, a single chain Fv (scFv) or a single domain antibody against IL-21.

In some embodiments, the cytokine is an IL-2 agonist polypeptide; and wherein the masking moiety is a Fab, a single chain Fv (scFv), or a single domain antibody against IL-2.

In some embodiments, the cytokine is an IL-15 agonist polypeptide; and wherein the masking moiety is a Fab, a single chain Fv (scFv), or a single domain antibody against IL-15. In some embodiments, the cytokine moiety comprises an IL-15 agonist polypeptide, wherein the chimeric molecule further comprises the sushi domain of IL-15 receptor α (IL-15Rα sushi domain).

In some embodiments, the IL-21 agonist polypeptide comprises an amino acid sequence as SEQ ID NO: 1, or at least 90% identical as that of SEQ ID NO: 1. In some embodiments, the masking moiety is IL-21Rα-ECD or a functional analog thereof, which comprises an amino acid sequence selected from SEQ ID NO: 12, 63-72, and 73, or at least 90% identical as that of SEQ ID NO: 12. In some embodiments, the masking moiety is an scFv which binds to human IL-21; and wherein the scFv comprises an amino acid sequence of SEQ ID NO: 20 or 21.

In some embodiments, the IL-2 agonist polypeptide comprises an amino acid sequence of SEQ ID NO: 6, or 61, or at least 95% identical as that of SEQ ID NO: 6, or 61. In some embodiments, the masking moiety comprises the IL-2Rβ-ECD or a functional analog thereof, wherein the IL-2-Rβ-ECD comprises an amino acid sequence of SEQ ID NO: 11 or at least 95% identical as that of SEQ ID NO: 11. In some embodiments, the masking moiety is an scFv which binds to IL-2, wherein the scFv interferes with the interaction between IL-2 and IL-2Rα. In some embodiments, the masking moiety is an scFv which binds to IL-2, wherein the scFv interferes with the interaction between IL-2 and IL-2Rβ. In some embodiments, the masking moiety is scFv which binds to IL-2, wherein the scFv interferes with the interaction between IL-2 and IL-2Rγ. In some embodiments, the masking moiety is scFv which binds to IL-2, wherein the scFv comprises the same heavy chain and light chain CDRs as that of hybridoma 4E12B2D10. In some embodiments, the masking moiety is an scFv which binds to IL-2, wherein the scFv comprises an amino acid sequence of SEQ ID NO: 22, 23, or at least 95% identical as that of SEQ ID NO: 22, 23.

In some embodiments, the IL-15 agonist polypeptide comprises an amino acid sequence of SEQ ID NO: 7, or at least 95% identical as that of SEQ ID NO: 7. In some embodiments, the IL-15 prodrug further comprises a sushi domain, which comprises an amino acid sequence of SEQ ID NO: 8, or at least 95% identical as that of SEQ ID NO: 8. In some embodiments, the masking moiety comprises the IL-2Rβ-ECD or a functional analog thereof, wherein the IL-2-Rβ-ECD comprises an amino acid sequence of SEQ ID NO: 11 or at least 95% identical as that of SEQ ID NO: 11. In some embodiments, the masking moiety is an scFv which binds to IL-15, wherein the scFv interferes with the interaction between IL-15 and IL-2Rβ. In some the embodiments, the masking moiety is an scFv which binds to IL-15, wherein the scFv interferes with the interaction between IL-15 and IL-2Rγ. In some embodiments, the masking moiety is scFv which binds to IL-15, wherein the scFv comprises the same heavy chain and light chain CDRs as that of the IL-15 antibody 146B7, 146H5, or 404E4. In some embodiments, the masking moiety is scFv which binds to IL-15, wherein the scFv comprises an amino acid sequence of SEQ ID NO: 18 or 19.

In some embodiments, the human IL-21 agonist polypeptide comprises one or more mutations at position(s) selected from D18, Q19, E109, and K117 (numbering according to SEQ ID NO: 1). In particular embodiments, the human IL-21 agonist polypeptide comprises an amino acid sequence selected from SEQ ID NOs: 1, 2, 3, 4, and 5.

In some embodiments of the present prodrugs, the cytokine moieties and the masking moieties are fused to the carrier moieties through a noncleavable peptide linker, such as one selected from SEQ ID NOs: 27-34.

In some embodiments of the present prodrugs, the carrier moiety is an IgG1 antibody that comprises mutations L234A and L235A (“LALA”) (EU numbering) or an IgG4 antibody that comprises mutations S228P/L234A/L235A (PAA). Other mutations which lead to the reduced Fc functionality, such as the ones described by Tam et al., Antibodies (2017) 6(12):1-34, can also be introduced when the Fc domain or the Fc of an antibody is used as the carrier moiety.

In particular embodiments, the carrier moiety is an antibody comprising knobs-into-holes mutations, wherein the cytokine moieties and the masking moieties are fused to the different heavy chains of the antibody. In certain embodiments, the knobs-into-holes mutations comprise a T366Y “knob” mutation on a polypeptide chain of the Fc domain or a heavy chain of the antibody, and a Y407T “hole” mutation in the other polypeptide of the Fc domain or the other heavy chain of the antibody (EU numbering). In certain embodiments, the knobs-into-holes mutations comprise Y349C and/or T366W mutations in the CH3 domain of the “knob chain” and E356C, T366S, L368A, and/or Y407V mutations in the CH3 domain of the “hole chain” (EU numbering).

In some embodiments, the carrier moiety is an antibody or an antigen-binding fragment thereof that specifically binds to one or more antigens selected from guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, CD47, signal-regulatory protein alpha (SIRPα), PD1, Claudin 18.2, Claudin 6, 5T4, BCMA, PD-L1, PD-1, fibroblast activation protein alpha (FAPalpha), the melanoma-associated chondroitin sulfate proteoglycan (MCSP), and epithelial cellular adhesion molecule (EPCAM). In specific embodiments, the carrier moiety is an antibody or fragment thereof which binds to FAPalpha or 5T4.

In another aspect, the present disclosure provides polynucleotides encoding the present prodrugs or fusion molecules, expression vectors comprising the polynucleotides, and host cells (e.g., mammalian host cells such as CHO, NS0 cells, and 293T cells) comprising the expression vectors. The present disclosure also provides methods for making the present prodrugs or fusion molecules, comprising culturing the mammalian host cells under conditions that allow expression of the prodrugs or fusion molecules and isolating the prodrugs or fusion molecules.

The present disclosure also provides a method of treating a cancer or an infectious disease or stimulating the immune system in a patient (e.g., human patient) in need thereof, comprising administering to the patient a therapeutically effective amount of the cytokine prodrug, or the pharmaceutical composition of the present disclosure. The patient may have, for example, a viral infection (e.g., HIV, HBV, HCV, or HPV infection), or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer. Also provided herein are cytokine prodrugs for use in treating a cancer or an infectious disease or stimulating the immune system in the present method; use of a cytokine prodrug for the manufacture of a medicament for treating a cancer or an infectious disease or stimulating the immune system in the present method; and articles of manufacture (e.g., kits) comprising one or more dosing units of the present cytokine prodrug.

Also provided herein are prodrugs for use in treating a cancer or an infectious disease or stimulating the immune system in the present method; use of a prodrug for the manufacture of a medicament for treating a cancer or an infectious disease or stimulating the immune system in the present method; and articles of manufacture (e.g., kits) comprising one or more dosing units of the present prodrugs.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTIONS OF THE DRAWINGS

FIGS. 1A-E illustrate a heterodimeric cytokine prodrug wherein the carrier is an antibody. FIG. 1A illustrates a prodrug wherein the carrier is a typical antibody with two light chains and two heavy chains. FIG. 1B illustrates a prodrug with a bispecific antibody as the carrier, wherein each antigen-binding moiety is monomeric with one antigen-binding moiety comprising a Fab domain and the second antigen-binding moiety comprising an scFv or a single domain antibody. FIG. 1C illustrates a prodrug with a bispecific antibody as the carrier, wherein each antigen-binding moiety is dimeric. FIG. 1D illustrates a prodrug with a dimeric Fc domain fused to a Fab domain on one of its polypeptide chains and to a cytokine peptide on the other polypeptide chain. The cytokine is fused via its N-terminus to a masking moiety. FIG. 1E illustrates a prodrug with a dimeric Fc domain fused to a Fab domain on one of its polypeptide chains and to a cytokine peptide on the other polypeptide chain. The masking moiety is fused via its N-terminus to a cytokine peptide.

FIG. 2 shows the binding of PD-1 antibody and Fc-IL-21 fusion molecules and control molecules to Mino cells. The binding was analyzed by FACS.

FIG. 3 shows the results of a NK-92 cell-based biological activity assay of IL-21 prodrugs prior to and after activation by protease MMP2 and the control molecules. PW04-38 αPD1-IL21-a* and PW09-16 αPD1-IL21-a* are two batches of the same molecule, which is an anti-PD-1 antibody-based IL-21 with IL-21R α-ECD as the masking moiety; PW05-68 αPD1-IL21-scFv is a PD-1 antibody-based IL-21 prodrug with the scFv as the masking moiety. A first control molecule PW04-67 αPD1-IL21 no mask is an anti-PD-1 antibody-IL-21 fusion molecule without a mask and having a wild-type IL-21. Another control molecule, PW09-02 αPD1-IL21muteinR9ER76A is an anti-PD-1 antibody-IL-21 fusion molecule without a mask and with an IL-21 mutein with R9E and R76A amino acid substitutions (numbering according to SEQ ID NO: 1). PW04-38 αPD1-IL21-a* activated and PW09-16 αPD1-IL21-a* activated are both the anti-PD-1 antibody-IL-21 wild type fusion molecule whose mask has been cleaved with a protease.

FIGS. 4A and 4B show the results of the Mino cell-based biological activity assay of PD-1-IL-21 prodrugs prior to and after activation by protease MMP2 and the control molecules. FIG. 4A shows the results after 72 hours of incubation of the cytokine fusion molecules with the Mino cells prior to the analysis. FIG. 4B shows the results after 120 hours of incubation prior to the analysis.

FIGS. 5A, 5B, and 5C show the results of Ki67 activation of the CD4+ T cells in PBMC after treated with an anti-PD-1 antibody (PD-1), an Fc-IL-15 fusion molecule masked with an scFv (215P), anti-PD1-antibody-IL-15 fusion molecule masked with an scFv (215γ), an Fc-IL-15 fusion molecule with no mask (215β ref), and an PD1-antibody-IL-15 fusion molecule with no mask (PD1/IL15 mutein (M2)). FIG. 5A shows the results of Ki67 activation of CD4+ T cells without PD-1 expression. FIG. 5B shows the results of Ki67 activation of CD4+ T cells with PD-1 expression. FIG. 5C shows the EC50 values of the testing articles with CD4+ T cells without and with PD-1 expression. It also shows the fold of changes of the EC50 values between the CD4+ T cells without and with PD-1 expression.

FIGS. 6A and 6B show the results of Ki67 activation of the CD4+ T cells in PBMC after treatment with an anti-PD-1 antibody (PD-1), a negative control (an IgG1 antibody), and an anti-PD1-antibody-IL-2 mutein fusion masked with IL-2 receptor β extracellular domain (IL-2Rβ-ECD) (PD-1/IL2V*). FIG. 6A shows the results of Ki67 activation of CD4+ T cells without PD-1 expression. FIG. 6B shows the results of Ki67 activation of CD4+ T cells with PD-1 expression.

FIG. 7 illustrates a novel mechanism of prodrug activation through “cis-binding.” The target cells (right) express both the antigen targeted by the carrier moiety and the receptor for the cytokine moiety. Binding of the prodrug to the antigen on the cell surface brings the prodrug to the proximity of the cytokine receptor on the cell, allowing the cytokine receptor to compete effectively with and displace the masking moiety, leading to cellular signaling by the bound cytokine receptor. No protease cleavage of the masking moiety is necessary to remove the mask and activate the prodrug. On cells expressing the cytokine receptor but not the antigen (left), the cytokine moiety of the prodrug remains masked and inactive.

### DETAILED DESCRIPTION OF THE INVENTION

As used herein and in the appended claims, the singular forms “a,” “or,” and “the” include plural referents unless the context clearly dictates otherwise.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, a polyclonal antibody, a multi-specific antibody, a dual-specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, an scFv, a single domain antibody (dAb), or a diabody, a single chain antibody, and an Fc-containing polypeptide such as an immunoadhesin). In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

As used herein, the phrase “against,” “binding to,” or “specifically binding to” in the context of the interaction between two molecules is intended to mean that the binding has a K_{D} not more than 1000 nM (e.g., not more than 100, 10, or 1 nM; such as less than 1 nM, 1-10 nM, 10-100 nM, or 100-1000 nM) as measured by common methods in the art (e.g., surface plasmon resonance, ELISA, and the like).

The term “cytokine agonist polypeptide” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine antagonist,” “masking moiety,” or “cytokine mask” refers to a moiety (e.g., a polypeptide) that binds to a cytokine and thereby inhibits the cytokine from binding to its receptor on the surface of a target cell and/or exerting its biological functions while being bound by the antagonist or mask. Examples of a cytokine antagonist or mask include, without limitations, a polypeptide derived from an extracellular domain of the cytokine's natural receptor that makes contact with the cytokine.

The term “effective amount” or “therapeutically effective amount” refers to an amount of a compound or composition sufficient to treat a specified disorder, condition, or disease (e.g., ameliorate, palliate, lessen, and/or delay one or more of its symptoms). In reference to a disease such as cancer, an effective amount may be an amount sufficient to delay cancer development or progression (e.g., decrease tumor growth rate, and/or delay or prevent tumor angiogenesis, metastasis, or infiltration of cancer cells into peripheral organs), reduce the number of epithelioid cells, cause cancer regression (e.g., shrink or eradicate a tumor), and/or prevent or delay cancer occurrence or recurrence. An effective amount can be administered in one or more doses.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “prodrug” refers to a therapeutic molecule that is inactive or has low activity in vitro or in the circulation while its activity is increased at the site of a disease.

The term “subject” refers to a mammal and includes, but is not limited to, a human, a pet (e.g., a canine or a feline), a farm animal (e.g., cattle or horse), a rodent, or a primate.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some, or all the properties of the various embodiments described herein may be combined to form other embodiments of the present invention. The section headings used herein are for organizational purposes only and are not to be construed as limiting the subject matter described thereunder.

### I. Cytokine Prodrugs

The present disclosure provides cytokine prodrugs that become more active at the site of a disease. The prodrugs comprise a cytokine agonist polypeptide (cytokine moiety) a carrier moiety, and a masking moiety. The cytokine moiety is linked, with or without a peptide linker (e.g., a noncleavable peptide linker), to the carrier moiety and is masked (bound) by the masking moiety. The masking moiety may be linked to the cytokine moiety or to the carrier moiety with or without a peptide linker (e.g., a noncleavable peptide linker). Alternatively, the cytokine moiety is linked, with or without a peptide linker (e.g., a noncleavable peptide linker), to the masking moiety, which in turn is linked, with or without a peptide linker (e.g., a noncleavable peptide linker), to the carrier moiety.

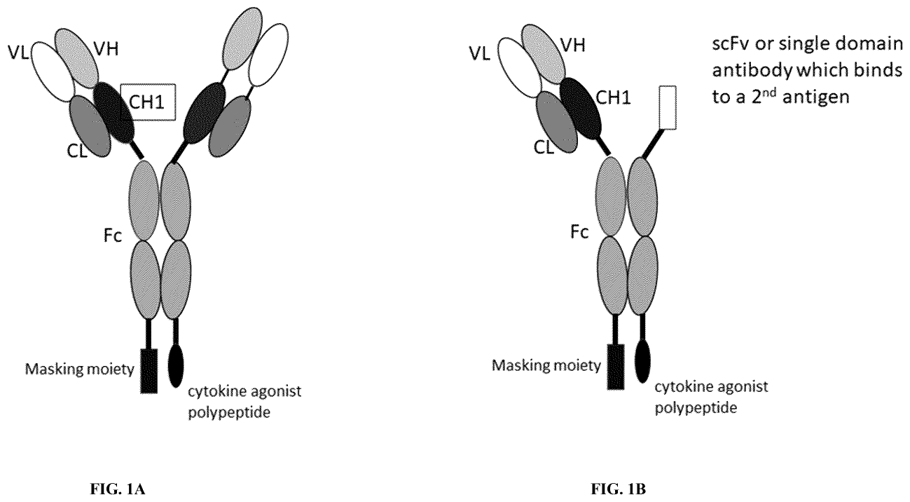

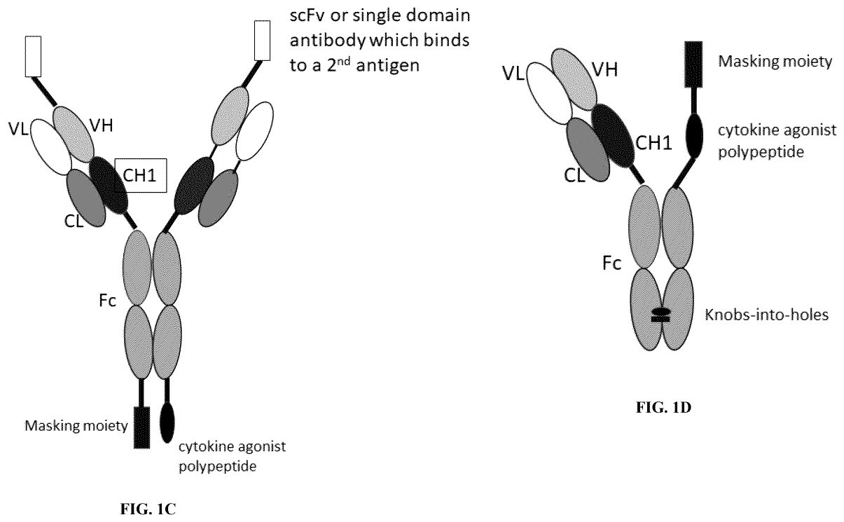

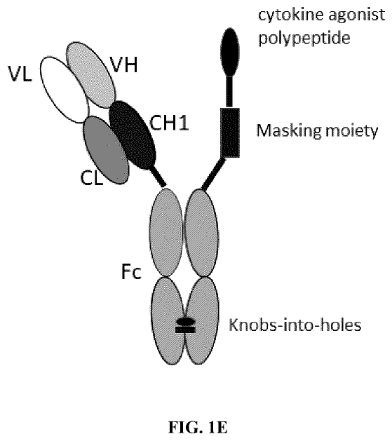

The carrier moiety comprises an antigen-binding domain that binds to an antigen on a target cell (e.g., a tumor cell or an immune cell). In some embodiments, the carrier comprises an antibody. See, e.g., FIG. 1A. In some embodiments, the carrier moiety comprises a bispecific antibody, which binds to two different antigens, as illustrated on FIGS. 1B and 1C.

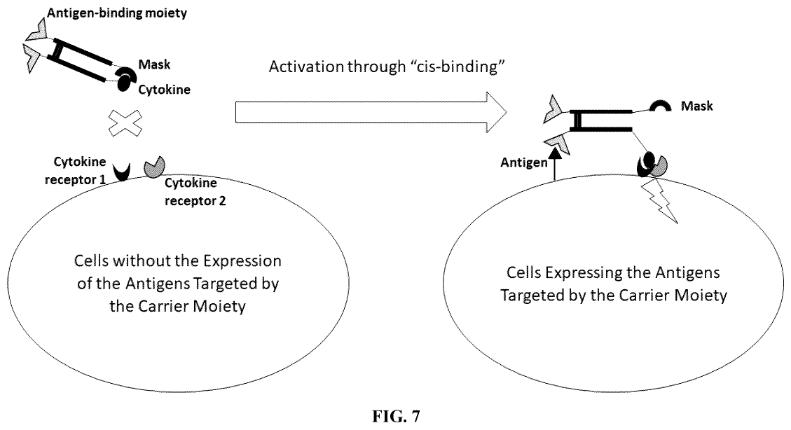

The masking moiety may comprise, for example, an extracellular domain (ECD) of a cytokine receptor, where the ECD is linked to the cytokine moiety (see, e.g., FIGS. 1D and 1E) and/or to the carrier moiety through a non-cleavable linker (see, e.g., FIGS. 1A-C and 1E). The mask binds to and inhibits the cytokine moiety's biological functions. These prodrugs can engage the target cell via “cis-binding” of a cytokine receptor and an antigen expressed on the cell surface, leading to increased activity of the prodrug without cleavage and removal of the masking moiety. The cytokine moiety of the prodrugs may increase in activity at a target site (e.g., at a tumor site or the surrounding environment), where both the antigen targeted by the carrier and a receptor of the cytokine are expressed on the same cell. Examples of such prodrugs are illustrated in FIGS. 1A-E and the “cis-binding” mechanism of action is illustrated in FIG. 7.

### A. Cytokine Moieties of the Prodrugs

In some embodiments, the cytokine moiety comprises an IL-2 agonist polypeptide. In some embodiments, the IL-2 agonist polypeptide is an IL-2 mutein having mutations R38S/F42A/Y45A/E62A or F42A/Y45A/L72G (numbering according to SEQ ID NO: 6). In some embodiments, the IL-2 mutein of the present disclosure may comprise a mutation at T3, D20, K35, R38, F42, F44, Y45, E62, E68, L72, A73, N88, N90, C125, and Q126 (numbering according to SEQ ID NO: 6). In certain embodiments, the novel IL-2 mutein comprises mutations at R38, F42, Y45, and A73 (numbering according to SEQ ID NO: 6).

In some embodiments, the cytokine moiety of the prodrug comprises an IL-15 agonist polypeptide. In some embodiments, the human IL-15 polypeptide comprises one or more mutations selected from N1A, N1D, N4A, N4D, I6T, S7A, D8A, D8T, D8E, D8N, K10A, K10D, K11A, K11D, E46, V49, L45, S51, L52, D61A, D61N, T62L, T62A, E64A, E64L, E64K, E64Q, N65A, N65L, N65D, L66D, L66E, I67D, I67E, I68S, I68E, L69S, L69E, N72A, N72D, V63E, V63D, L66E, L66D, I67E, I67D, Q108E, N112A, N1D/D61N, N1D/E64Q, N4D/D61N, N4D/E64Q, D8N/D61N, D8N/E64Q, D61N/E64Q, E64Q/Q108E, N1D/N4D/D8N, D61N/E64Q/N65D, N1D/D61N/E64Q, N1D/Q108E, N1D/D61N/E64Q/Q108E, N4D/D61N/E64Q/Q108E, and D30N/E64Q/N65D (numbering according SEQ ID NO: 7). In some embodiments, the IL-15 prodrug further comprises a sushi domain. In some embodiments, the sushi domain comprises an amino acid sequence of SEQ ID NO: 8 or at least 95% identical as that of SEQ ID NO: 8.

In some embodiments, the cytokine moiety comprises an IL-21 agonist polypeptide. In some embodiments, the IL-21 agonist polypeptide may be a wildtype IL-21 polypeptide such as a wildtype human IL-21 (e.g., SEQ ID NO: 1), or an IL-21 mutein derived from a human IL-21, e.g., one with an amino acid sequence selected from SEQ ID NOs: 2-5. The IL-21 mutein may have significantly reduced affinity for IL-21Rα or IL-21RαRγ, as compared to wild type IL-21. In some embodiments, the IL-21 mutein has a binding affinity for the high-affinity IL-2Rα that is 5 times, 10 times, 20 times, 50 times, 100 times, 300 times, 500 times, 1,000 times, or 10,000 times lower compared to wild type IL-21. Unless otherwise indicated, all residue numbers in IL-21 and IL-21 muteins described herein are in accordance with the numbering in SEQ ID NO: 1.

### B. Masking Moieties of the Prodrugs

The masking moiety, in the present prodrug may comprise a peptide or an antibody or antibody fragment that binds to the cytokine moiety in the prodrug, masking the cytokine moiety and inhibiting its biological functions. In some embodiments, the masking moiety is operationally linked to the rest of the prodrug through a non-cleavable peptide linker.

By way of example, the prodrug comprises an IL-2 agonist polypeptide and the masking moiety comprises a peptide or antibody, or antigen-binding fragment thereof, that binds IL-2 and interferes with the binding of the IL-2 to its cognate receptors. In some embodiments, the masking moiety reduces biological activities of the IL-2 moiety while masked. In some embodiments, the IL-2 antagonist comprises an IL-2Rβ or IL-2Rγ extracellular domain or its functional analog, such as one derived from human IL-2Rβ or human IL-2Rγ (e.g., SEQ ID NO: 11 or 13). In some embodiments, the IL-2 masking moiety comprises a peptide identified through a peptide library screening. In some embodiments, the IL-2 masking moiety comprises an antibody or fragment thereof that blocks the binding of IL-2 or IL-2 muteins to an IL-2 receptor. In some embodiments, the masking moiety comprises an scFv of an antibody against IL-2. In some embodiments, the masking moiety comprises an amino acid sequence of SEQ ID NO: 22 or 23, or at least 95% identical as that of SEQ ID NO: 22 or 23.

By way of another example, the prodrug comprises an IL-21 agonist polypeptide and the masking moiety comprises a peptide or antibody, or antigen-binding fragment thereof, that binds IL-21 and interferes with the binding of the IL-21 to its cognate receptors. In some embodiments, the masking moiety reduces biological activities of the IL-21 moiety while masked. In some embodiments, the IL-21 antagonist comprises an IL-21Rα or IL-21Rγ extracellular domain or its functional analog, such as one derived from human IL-21Rα or human IL-21Rγ (e.g., SEQ ID NO: 12 or 13). In some embodiments, the IL-21 masking moiety comprises a peptide identified through a peptide library screening. In some embodiments, the IL-21 masking moiety comprises an antibody or fragment thereof that blocks the binding of IL-21 or IL-21 muteins to an IL-21 receptor. In some embodiments, the masking moiety comprises an scFv of an antibody against IL-21. In some embodiments, the masking moiety comprises an amino acid sequence of SEQ ID NO: 20 or 21, or at least 95% identical as that of SEQ ID NO: 20 or 21.

By way of another example, the prodrug comprises a masking moiety of any cytokine disclosed herein, including, but not limited to a cytokine selected from IL-7 agonist polypeptide, an IL-9 agonist polypeptide, an IL-15 agonist polypeptide, an IL-1α agonist polypeptide, an IL-1β agonist polypeptide, an IL-4 agonist polypeptide, an IL-5 agonist polypeptide, an IL-6 agonist polypeptide, an IL-8 agonist polypeptide, an IL-10 agonist polypeptide, an IL-12 agonist polypeptide, an IL-17 agonist polypeptide, an IL-18 agonist polypeptide, an IL-22 agonist polypeptide, an IL-23 agonist polypeptide, an IL-31 agonist polypeptide, an IL-33 agonist polypeptide, an IL-36 agonist polypeptide, an Interferon-alpha agonist polypeptide, an interferon gamma agonist polypeptide, a 4-1BB ligand, an OX-40 ligand, and a CD-40 ligand.

### C. Carrier Moieties of the Prodrugs

The carrier moieties of the present prodrugs comprise an antigen-binding domain and optionally other domains. The carrier moiety improves the PK profiles such as serum half-life of the cytokine agonist polypeptide, and also targets the cytokine agonist polypeptide to a target site in the body, such as a tumor site.

### 1. Antigen-Binding Domains of Carrier Moieties

The carrier moiety comprises an antigen-binding domain and may be an antibody or an antigen-binding fragment thereof, or an immunoadhesin. In some embodiments, the antigen-binding carrier moiety is a full-length antibody with two heavy chains and two light chains, a Fab fragment, a Fab′ fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. The antigen-binding moiety may provide additional and potentially synergetic therapeutic efficacy to the cytokine agonist polypeptide.

The cytokine agonist polypeptide and its mask may be fused to the N-terminus or C-terminus of the light chain(s) and/or heavy chain(s) of the antigen-binding moiety. By way of example, the IL-21 agonist polypeptide and its mask may be fused to the antibody heavy chain or an antigen-binding fragment thereof or to the antibody light chain or an antigen-binding fragment thereof. In some embodiments, one terminus of the IL-21 agonist polypeptide is fused to the C-terminus of one or both heavy chains of an antibody, and the IL-21 mask is fused to the other terminus of the IL-21 agonist polypeptide through a non-cleavable peptide linker. In some embodiments, the IL-21 agonist polypeptide is fused to the C-terminus of one of the heavy chains of an antibody, and the IL-21 mask is fused to the C-terminus of the other heavy chain of the antibody through a non-cleavable peptide linker, wherein the two heavy chains contain mutations that allow the specific pairing of the two different heavy chains.

Strategies for forming heterodimers are well known (see, e.g., Spies et al., Mol Imm. (2015) 67(2)(A):95-106). For example, the two heavy chain polypeptides in the prodrug may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. No. 8,642,745). For example, the Fc domain of the antibody may comprise a T366W mutation in the CH3 domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech (1998) 16:677-81). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antigen-binding moiety in the prodrug is an antibody that comprises L234A and L235A (“LALA”) mutations in its Fc domain. The LALA mutations eliminate complement binding and fixation as well as Fcγ dependent ADCC (see, e.g., Hezareh et al. J. Virol. (2001) 75(24):12161-8). In further embodiments, the LALA mutations are present in the antibody moiety in addition to the knobs-into-holes mutations.

In some embodiments, the antigen-binding moiety is an antibody that comprises the M252Y/S254T/T256E (“YTE”) mutations in the Fc domain. The YTE mutations allow the simultaneous modulation of serum half-life, tissue distribution and activity of IgG₁ (see Dall' Acqua et al., J Biol Chem. (2006) 281:23514-24; and Robbie et al., Antimicrob Agents Chemother. (2013) 57(12):6147-53). In further embodiments, the YTE mutations are present in the antibody in addition to the knobs-into-holes mutations. In particular embodiments, the antibody has YTE, LALA and knobs-into-holes mutations or any combination thereof.

In particular embodiments, the antigen-binding moiety is an antibody, or antigen-binding fragment thereof, that binds to an antigen on the surface of a target cell, such as an immune cell. Immune cells are well known in the art. Non-limiting examples of immune cells include T cells, NK cells, and macrophages. The antigen-binding moiety may have the ability to activate the immune cell and enhance its anti-cancer activity. The antibody may or may not have ADCC activity. The antigen-binding moiety may also be further conjugated to a cytotoxic drug. In some embodiments, the antigen-binding moiety may bind to PD-1, LAG-3, TIM-3, TIGIT, CTLA-4, or TGF-beta. In other embodiments, the antigen-binding moiety may bind to an antigen on the surface of a target cell, such as a tumor cell. For example, the antigen-binding moiety may bind to FAP alpha, 5T4, Trop-2, PD-L1, HER-2, EGFR, Claudin 18.2, DLL-3, GCP3, or carcinoembryonic antigen (CEA).

In some embodiments, the antigen-binding moiety binds to guanyl cyclase C (GCC), carbohydrate antigen 19-9 (CA19-9), glycoprotein A33 (gpA33), mucin 1 (MUC1), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostates-specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, CD47, signal-regulatory protein alpha (SIRPα), Claudin 18.2, Claudin 6, BCMA, or EPCAM. In some embodiments, the antigen-binding moiety binds to an epidermal growth factor (EGF)-like domain of DLL3. In some embodiments, the antigen-binding moiety binds to a Delta/Serrate/Lag2 (DSL)-like domain of DLL3. In some embodiments, the antigen-binding moiety binds to an epitope located after the 374th amino acid of GPC3. In some embodiments, the antigen-binding moiety binds to a heparin sulfate glycan of GPC3. In some embodiments, the antigen-binding moiety binds to Claudin 18.2 and does not bind to Claudin 18.1. In some embodiments, the antigen-binding moiety binds to Claudin 18.1 with at least 10 times weaker binding affinity than to Claudin 18.2.

Exemplary antigen-binding moieties include trastuzumab, rituximab, brentuximab, cetuximab, panitumumab, GC33 (or a humanized version thereof), anti-EGFR antibody mAb806 (or a humanized version thereof), anti-dPNAG antibody F598, and antigen-binding fragments thereof. In some embodiments, the antigen-binding moiety has at least 90%, 91%, 92%, 93%, 94%, 95%, 96%, 97%, 98%, or 99% identity to trastuzumab, rituximab, brentuximab, cetuximab, or panitumumab, GC33 (or a humanized version thereof), anti-EGFR antibody mAb806 (or a humanized version thereof), anti-dPNAG antibody F598, or a fragment thereof. In some embodiments, the antigen-binding moiety comprises an antibody heavy chain with at least 90%, 91%, 92%, 93%, 94%, 95%, 96%, 97%, 98%, or 99% identity to the antibody heavy chain of trastuzumab, rituximab, brentuximab, cetuximab, panitumumab, GC33 (or a humanized version thereof), anti-EGFR antibody mAb806 (or a humanized version thereof), anti-dPNAG antibody F598, or a fragment thereof. In some embodiments, the antigen-binding moiety has an antibody light chain with at least 90%, 91%, 92%, 93%, 94%, 95%, 96%, 97%, 98%, or 99% identity to the antibody light chain of trastuzumab, rituximab, brentuximab, cetuximab, panitumumab, GC33 (or a humanized version thereof), anti-EGFR antibody mAb806 (or a humanized version thereof), anti-dPNAG antibody F598, or a fragment thereof. The antigen-binding moiety is fused to an IL-2 agonist polypeptide. In some embodiments, the antigen-binding moiety comprises the six complementarity-determining regions (CDRs) of trastuzumab, rituximab, brentuximab, cetuximab, panitumumab, GC33, anti-EGFR antibody mAb806, or anti-dPNAG antibody F598.

A number of CDR delineations are known in the art and are encompassed herein. A person of skill in the art can readily determine a CDR for a given delineation based on the sequence of the heavy or light chain variable region. The “Kabat” CDRs are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, J. Mol. Biol. (1987) 196:901-917). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid numbers in antibodies refer to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 | H31-H35B | H26-H35B | H26-H32 | H30-H35B |
| (Kabat nos.) | | | | |
| VH-CDR1 | H31-H35 | H26-H35 | H26-H32 | H30-H35 |
| (Chothia nos.) | | | | |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the antigen-binding moiety binds to PDL1, and comprises a light chain having an amino acid sequence with at least 90%, 91%, 92%, 93%, 94%, 95%, 96%, 97%, 98%, or 99% identity to SEQ ID NO: 45, or a fragment thereof, and a heavy chain having an amino acid sequence with at least 90%, 91%, 92%, 93%, 94%, 95%, 96%, 97%, 98%, or 99% identity to SEQ ID NO: 46, or a fragment thereof. In some embodiments, the antigen-binding domain comprises CDR1, CDR2, and CDR3 from SEQ ID NO: 45, and CDR1, CDR2, and CDR3 from SEQ ID NO: 46.

In some embodiments, the antigen-binding moiety binds to PD-1, and comprises a light chain having an amino acid sequence with at least 90%, 91%, 92%, 93%, 94%, 95%, 96%, 97%, 98%, or 99% identity to SEQ ID NO: 44, or a fragment thereof, and a heavy chain having an amino acid sequence with at least 90%, 91%, 92%, 93%, 94%, 95%, 96%, 97%, 98%, or 99% identity to SEQ ID NO: 47, or a fragment thereof. In some embodiments, the antigen-binding domain comprises CDR1, CDR2, and CDR3 from SEQ ID NO: 44, and CDR1, CDR2, and CDR3 from SEQ ID NO: 47.

In some embodiments, the antigen-binding moiety binds to PD-1, and comprises a light chain having an amino acid sequence with at least 90%, 91%, 92%, 93%, 94%, 95%, 96%, 97%, 98%, or 99% identity to SEQ ID NO: 48, or a fragment thereof, and a heavy chain having an amino acid sequence with at least 90%, 91%, 92%, 93%, 94%, 95%, 96%, 97%, 98%, or 99% identity to SEQ ID NO: 49, or a fragment thereof. In some embodiments, the antigen-binding domain comprises CDR1, CDR2, and CDR3 from SEQ ID NO: 48, and CDR1, CDR2, and CDR3 from SEQ ID NO: 49.

In some embodiments, the antigen-binding moiety comprises one, two, or three antigen-binding domains. For example, the antigen-binding moiety is bispecific and binds to two different antigens selected from the group consisting of CD3, HER2, HER3, EGFR, 5T4, FAP alpha, Trop-2, GPC3, VEGFR2, Claudin 18.2 and PD-L1. In some embodiments, the bispecific antigen-binding moiety binds to two different epitopes of HER2. In other embodiments, the antigen-binding moiety is bispecific and binds to two different antigens selected from PD-1, PD-L1, CTLA-4, CD47, CD3, TIM-3, LAG-3 and TIGIT.

### 2. Other Domains of Carrier Moieties

The carrier moieties may also comprise other domains that are not antigen-binding. For example, an antibody Fc domain (e.g., a human IgG₁, IgG₂, IgG₃, or IgG₄ Fc), a polymer (e.g., PEG), an albumin (e.g., a human albumin) or a fragment thereof, or a nanoparticle can be used.

By way of example, the cytokine agonist polypeptide (e.g., IL-2, IL-21, IL-5, or any cytokine disclosed herein) and its antagonist may be fused to an antibody Fc domain, forming an Fc fusion protein. In some embodiments, the cytokine agonist polypeptide is fused (directly or through a peptide linker) to the C-terminus or N-terminus of one of the Fc domain polypeptide chains, and the cytokine mask is fused to the corresponding C-terminus or N-terminus of the other Fc domain polypeptide chain through a cleavable peptide linker, wherein the two Fc domain polypeptide chains contain mutations that allow the specific pairing of the two different Fc chains. In some embodiments, the Fc domain comprises the holes-into-holes mutations described above. In further embodiments, the Fc domain may comprise also the YTE and/or LALA mutations described above.

The carrier moiety of the prodrug may comprise an albumin (e.g., human serum albumin) or a fragment thereof. In some embodiments, the carrier moiety comprises an albumin fragment (e.g., a human serum albumin fragment) that is about 10 or more, 20 or more, 30 or more 40 or more, 50 or more, 60 or more, 70 or more, 80 or more, 90 or more, 100 or more, 120 or more, 140 or more, 160 or more, 180 or more, 200 or more, 250 or more, 300 or more, 350 or more, 400 or more, 450 or more, 500 or more, or 550 or more amino acids in length. In some embodiments, the albumin fragment is between about 10 amino acids and about 584 amino acids in length (such as between about 10 and about 20, about 20 and about 40, about 40 and about 80, about 80 and about 160, about 160 and about 250, about 250 and about 350, about 350 and about 450, or about 450 and about 550 amino acids in length). In some embodiments, the albumin fragment includes the Sudlow I domain or a fragment thereof, or the Sudlow II domain or the fragment thereof.

### D. Linker Components of the Prodrugs

The cytokine agonist polypeptide may be fused to the carrier moiety with or without a peptide linker. The peptide linker is noncleavable. In some embodiments, the peptide linker is selected from SEQ ID NOs: 27-34. In particular embodiments, the peptide linker comprises the amino acid sequence GGGGSGGGGSGGGGS (SEQ ID NO: 30).

The masking moiety may be fused to the cytokine moiety or to the carrier through a non-cleavable linker or without a peptide linker.

### II. Pharmaceutical Compositions

Pharmaceutical compositions comprising the prodrugs and muteins (i.e., the active pharmaceutical ingredient or API) of the present disclosure may be prepared by mixing the API having the desired degree of purity with one or more optional pharmaceutically acceptable excipients (see, e.g., Remington's Pharmaceutical Sciences, 16th Edition., Osol, A. Ed. (1980)) in the form of lyophilized formulations or aqueous solutions. Pharmaceutically acceptable excipients (or carriers) are generally nontoxic to recipients at the dosages and concentrations employed, and include, but are not limited to: buffers containing, for example, phosphate, citrate, succinate, histidine, acetate, or another inorganic or organic acid or salt thereof, antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including sucrose, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, and acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may additionally comprise any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route.

In some embodiments, the pharmaceutical composition of the present disclosure is a lyophilized protein formulation. In other embodiments, the pharmaceutical composition may be an aqueous liquid formulation.

### III. Methods of Treatment

The cytokine prodrug and fusion molecules can be used to treat a disease, depending on the antigen bound by the antigen-binding domain. In some embodiments, the cytokine prodrug or fusion molecule is used to treat cancer. In some embodiments, the cytokine prodrug or fusion molecule is used to treat an infection.

In some embodiments, a method of treating a disease (such as cancer, a parasitic infection, a viral infection, or a bacterial infection) in a subject comprises administering to the subject an effective amount of a cytokine prodrug or a fusion molecule.

In some embodiments, the cancer is a solid cancer. In some embodiments, the cancer is a blood cancer or a solid tumor. Exemplary cancers that may be treated include, but are not limited to, leukemia, lymphoma, kidney cancer, bladder cancer, urinary tract cancer, cervical cancer, brain cancer, head and neck cancer, skin cancer, uterine cancer, testicular cancer, esophageal cancer, liver cancer, colorectal cancer, stomach cancer, squamous cell carcinoma, prostate cancer, pancreatic cancer, lung cancer such as non-small cell lung cancer, cholangiocarcinoma, breast cancer, and ovarian cancer.

In some embodiments, the cytokine prodrug or fusion molecule is used to treat a viral infection. In some embodiments, the virus causing the viral infection is hepatitis C virus (HCV), hepatitis B virus (HBV), human immunodeficiency virus (HIV), or human papilloma virus (HPV). In some embodiments, the antigen-binding moiety binds to a viral antigen.

In some embodiments, the cytokine prodrug or fusion molecule is used to treat a bacterial infection such as sepsis. In some embodiments, the bacteria causing the bacterial infection is drug-resistant bacteria. In some embodiments, the antigen-binding moiety binds to a bacterial antigen.

Generally, dosages and routes of administration of the present pharmaceutical compositions are determined according to the size and conditions of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, intracranially, or intraspinally. In some embodiments, the composition is administered to a subject intravenously.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the pharmaceutical composition is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 3, 4, 5, 7, 10, 15, or 20 or more doses).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

### IV. Exemplary Embodiments

Further, particular embodiments of the present disclosure are described as follows. These embodiments are intended to illustrate the compositions and methods described in the present disclosure and are not intended to limit the scope of the present disclosure.

1. A prodrug comprising a cytokine moiety, a masking moiety, and a carrier moiety,
wherein:
a) the masking moiety binds to the cytokine moiety and inhibits an intended biological activity of the cytokine;
b) the carrier moiety comprises an antigen binding moiety which binds to an antigen expressed on the surface of a cell; and
c) the masking moiety is linked directly or indirectly to the carrier moiety through a non-cleavable peptide linker or without a peptide linker;
   and wherein the activity of the prodrug is higher in stimulating a cell which expresses both the antigen targeted by the carrier moiety and the receptor(s) of the cytokine than its activity in stimulating a cell which expresses the receptor(s) of the cytokine but not the antigen targeted by the carrier moiety.

2. A prodrug comprising a cytokine moiety, a masking moiety, and a carrier moiety,
wherein:
a) the masking moiety binds to the cytokine moiety and inhibits an intended biological activity of the cytokine;
b) the carrier moiety comprises an antigen binding moiety which binds to an antigen expressed on the surface of a cell; and
c) the masking moiety is linked directly or indirectly to the carrier moiety through a non-cleavable peptide linker or without a peptide linker;
and wherein the prodrug is activated at a disease site where there are cells expressing both the antigen targeted by the carrier moiety and the receptor(s) of the cytokine, or a biological activity of the prodrug is increased by at least 2 folds, at least 5 folds, or at least 10 folds at a disease site where there are cells expressing both the antigen targeted by the carrier moiety and the receptor(s) of the cytokine.

3. The prodrug of embodiment 1 or 2, wherein the receptor of the cytokine moiety comprises two or more subunits.

4. The prodrug of embodiment 1 or 2, wherein the carrier comprises an antigen-binding moiety, wherein the antigen is expressed on an immune cell.

5. The prodrug of embodiment 1 or 2, wherein the carrier moiety comprises an antigen-binding moiety, wherein the antigen is selected from PD-1, PD-L1, CTLA-4, TIGIT, TIM-3, LAG-3, CD25, CD16a, CD16b, NKG2D, NKP44, NKP30, CD19, CD20, CD38, and BCMA.

6. The prodrug of any of embodiments 1-5, wherein the cytokine moiety comprises a cytokine selected from an IL-2 agonist polypeptide, an IL-7 agonist polypeptide, an IL-9 agonist polypeptide, an IL-15 agonist polypeptide, and an IL-21 agonist polypeptide.

7. The prodrug of any of embodiments 1-5, wherein the cytokine is selected from an IL-1α agonist polypeptide, an IL-1β agonist polypeptide, an IL-4 agonist polypeptide, an IL-5 agonist polypeptide, an IL-6 agonist polypeptide, an IL-8 agonist polypeptide, an IL-10 agonist polypeptide, an IL-12 agonist polypeptide, an IL-17 agonist polypeptide, an IL-18 agonist polypeptide, an IL-22 agonist polypeptide, an IL-23 agonist polypeptide, an IL-31 agonist polypeptide, an IL-33 agonist polypeptide, an IL-36 agonist polypeptide, an Interferon-alpha agonist polypeptide, an interferon gamma agonist polypeptide, a 4-1BB ligand, an OX-40 ligand, and a CD-40 ligand.

8. The prodrug of any of embodiments 1-7, wherein the masking moiety is an extracellular domain (ECD) of a receptor of the cytokine.

9. The prodrug of any of embodiments 1-6, wherein the cytokine is an IL-7 agonist polypeptide; and wherein the masking moiety is the extracellular domain of IL-7 receptor α (IL-7Rα ECD) or a functional analog thereof.

10. The prodrug of any of embodiments 1-6, wherein the cytokine is an IL-21 agonist polypeptide; and wherein the masking moiety is the extracellular domain of IL-21 receptor α (IL-21Rα ECD) or a functional analog thereof.

11. The prodrug of any of embodiments 1-6, wherein the cytokine is an IL-2 agonist polypeptide or an IL-15 agonist polypeptide; and wherein the masking moiety is the extracellular domain of IL-2 receptor β (IL-2Rβ ECD).

12. The prodrug of any of embodiments 1-6, wherein the cytokine is an IL-21 agonist polypeptide; and wherein the masking moiety is a Fab, a single chain Fv (scFv) or a single domain antibody against IL-21.

13. The prodrug of any of embodiments 1-6, wherein the cytokine is an IL-2 agonist polypeptide; and wherein the masking moiety is a Fab, a single chain Fv (scFv), or a single domain antibody against IL-2.

14. The prodrug of any of embodiments 1-6, wherein the cytokine is an IL-15 agonist polypeptide; and wherein the masking moiety is a Fab, a single chain Fv (scFv), or a single domain antibody against IL-15.

15. The prodrug of any of embodiments 1-6, 11 and 14, wherein the cytokine moiety comprises an IL-15 agonist polypeptide, wherein the fusion molecule further comprises the sushi domain of IL-15 receptor α (IL-15Rα sushi domain).

16. The prodrug of any of embodiment 10 or 12, wherein the IL-21 agonist polypeptide comprises an amino acid sequence as SEQ ID NO: 1, or at least 90% identical as that of SEQ ID NO: 1.

17. The prodrug of embodiment 10 or 16, wherein the masking moiety is IL-21Rα-ECD or a functional analog thereof, which comprises an amino acid sequence selected from SEQ ID NO: 12, 63-72, and 73, or at least 90% identical as that of SEQ ID NO: 12.

18. The prodrug of embodiment 12 or 16, wherein the masking moiety is an scFv which binds to human IL-21; and wherein the scFv comprises an amino acid sequence of SEQ ID NO: 20 or 21.

19. The prodrug of embodiment 11 or 13, wherein the IL-2 agonist polypeptide comprises an amino acid sequence of SEQ ID NO: 6, or 61, or at least 95% identical as that of SEQ ID NO: 6, or 61.

20. The prodrug of embodiment 11 or 14, wherein the IL-15 agonist polypeptide comprises an amino acid sequence of SEQ ID NO: 7, or at least 95% identical as that of SEQ ID NO: 7.

21. The prodrug of embodiment 11, 19, or 20, wherein the masking moiety comprises the IL-2Rβ-ECD or a functional analog thereof, wherein the IL-2-Rβ-ECD comprises an amino acid sequence of SEQ ID NO: 11 or at least 95% identical as that of SEQ ID NO: 11.

22. The prodrug of any of embodiments 6, 13 or 19, wherein the masking moiety is an scFv which binds to IL-2, wherein the scFv inhibits or interferes with the interaction between IL-2 and IL-2Rα, the interaction between IL-2 and IL-2Rβ, and/or the interaction between IL-2 and IL-2Rγ.

23. The prodrug of any of embodiments 6, 14, 15 or 20, wherein the masking moiety is an scFv which binds to IL-15, wherein the scFv inhibits or interferes with the interaction between IL-15 and IL-2Rβ, and/or IL-2 and IL-2Rγ.

24. The prodrug of any of embodiments 6, 12 or 16, wherein the masking moiety is an scFv which binds to IL-21, wherein the scFv inhibits or interferes with the interaction between IL-21 and IL-21Rα, and/or IL-21 and IL-2Rγ.

25. The prodrug of embodiment 6, wherein the masking moiety is an scFv which binds to a cytokine selected from IL-2, IL-7, IL-9, IL-15, or IL-21; wherein the scFv inhibits or interferes with the interaction between the cytokine and IL-2Rγ; and wherein the prodrug has a half-life in a non-human primate or a human that is at least 20 times, at least 50 times, at least 100 times, at least 150 times, or at least 200 times longer than that of the corresponding wild type cytokine.

26. A prodrug which comprises a carrier moiety, a cytokine moiety, and a masking moiety; wherein:
a) the cytokine moiety is selected from an IL-2 agonist polypeptide, an IL-7 agonist polypeptide, an IL-9 agonist polypeptide, an IL-15 agonist polypeptide, or an IL-21 agonist polypeptide;
b) the masking moiety is an scFv which binds to the cytokine moiety;
c) the scFv inhibits or interferes with the interaction between the cytokine and IL-2Rγ; and
d) wherein the prodrug has a half-life in a non-human primate or a human that is at least 20 times, at least 50 times, at least 100 times, at least 150 times, or at least 200 times longer than that of the wild type cytokine.

27. The prodrug of any of embodiments 22-25, wherein the scFv binds to the cytokine with a K_{D} of 1-10 nM.

28. The prodrug of any of embodiments 22-25, wherein the scFv binds to the cytokine with a K_{D} of 10-100 nM.

29. The prodrug of any of embodiments 22-25, wherein the scFv binds to the cytokine with a K_{D} of 100-1000 nM.

30. The prodrug of embodiment 13 or 19, wherein the masking moiety is scFv which binds to IL-2, wherein the scFv comprises the same heavy chain and light chain CDRs as that of hybridoma 4E12B2D10.

31. The prodrug of embodiment 13 or 19, wherein the masking moiety is scFv which binds to IL-2, wherein the scFv comprises an amino acid sequence of SEQ ID NO: 22 or 23, or at least 95% identical as that of SEQ ID NO: 22 or 23.

32. The prodrug of embodiment 14 or 20, wherein the masking moiety is scFv which binds to IL-15, wherein the scFv comprises the same heavy chain and light chain CDRs as that of the IL-15 antibody 146B7, 146H5, or 404E4.

33. The prodrug of embodiment 14 or 20, wherein the masking moiety is scFv which binds to IL-15, wherein the scFv comprises an amino acid sequence of SEQ ID NO: 18 or 19.

34. The prodrug of any of the embodiments 1-32, wherein the prodrug does not contain a cleavable peptide linker.

35. The prodrug of embodiment 1, wherein the prodrug comprises two light chains with an amino acid sequence of SEQ ID NO: 44, and two heavy chain polypeptide chains whose amino acid sequences respectively comprise
SEQ ID NOs: 35 and 36,
SEQ ID NOs: 37 and 36,
SEQ ID NOs: 37 and 38,
SEQ ID NOs: 39 and 41, or
SEQ ID NOs: 42 and 43.

36. The prodrug of embodiment 1, wherein the prodrug comprises two light chains with an amino acid sequence of SEQ ID NO: 50, and two heavy chain polypeptide chains whose amino acid sequences respectively comprise
SEQ ID NOs: 51 and 54,
SEQ ID NOs: 51 and 55,
SEQ ID NOs: 51 and 56,
SEQ ID NOs: 52 and 54,
SEQ ID NOs: 53 and 58,
SEQ ID NOs: 53 and 59, or
SEQ ID NOs: 52 and 57.

37. A pharmaceutical composition comprising the prodrug of any one of the embodiments 1-36 and a pharmaceutically acceptable excipient.

38. A polynucleotide or polynucleotides encoding the prodrug of any one of embodiments 1-36.

39. An expression vector or vectors comprising the polynucleotide or polynucleotides of embodiment 38.

40. A host cell comprising the vector(s) of embodiment 39.

41. A method of making the prodrug of any one of embodiments 1-36, comprising
culturing the host cell of embodiment 46 under conditions that allow expression of the prodrug, wherein the host cell is a mammalian cell, and
isolating the prodrug.

42. A method of treating a cancer, an autoimmune disease, or an infectious disease, or stimulating the immune system, in a patient in need thereof, comprising administering to the patient a therapeutically effective amount of the pharmaceutical composition of embodiment 37.

43. A prodrug of any one of embodiments 1-36, for use in treating a cancer, an autoimmune disease or an infectious disease, or stimulating the immune system, in a patient in need thereof.

44. Use of a prodrug of any one of embodiments 1-36, for the manufacture of a medicament for treating a cancer, an autoimmune disease, or an infectious disease, or stimulating the immune system, in a patient in need thereof.

45. The method of embodiment 41, the prodrug for use of claim 42 or the fusion molecule for use of claim 43, or the use of claim 44, wherein the patient has HIV, HBV, HCV, or HPV infection; an autoimmune disease selected from lupus, type I diabetes, psoriasis, dermatomyositis, GvHD, or rheumatoid arthritis; or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.

46. A prodrug which comprises a carrier moiety, a cytokine moiety, and a masking moiety; wherein:
a) the cytokine moiety is selected from an IL-2 agonist polypeptide, an IL-7 agonist polypeptide, an IL-9 agonist polypeptide, an IL-15 agonist polypeptide, or an IL-21 agonist polypeptide;
b) the masking moiety is an scFv which binds to the cytokine moiety;
c) the scFv inhibits or interferes with the interaction between the cytokine and IL-2Rγ; and
d) wherein the prodrug has an area under curve (AUC) in a non-human primate or a human that is at least 20 times, at least 50 times, at least 100 times, at least 150 times, or at least 200 times longer than that of the wild type cytokine.

In order that this invention may be better understood, the following examples are set forth. These examples are for purposes of illustration only and are not to be construed as limiting the scope of the invention in any manner.

### Examples

The materials and methods for the experiments described in Examples 1 and 2 are as follows.

### SEC-HPLC Analysis

SEC-HPLC was carried out using an Agilent 1100 Series of HPLC system with a TSKgel G3000SWXL column (7.8 mmIDX 30 cm, 5 μm particle size) ordered from Tosoh Bioscience. A sample of up to 100 μl was loaded. The column was run with a buffer containing 200 mM K₃PO₄, 250 mM KCl, pH 6.5. The flow rate was 0.5 ml/min. The column was run at room temperature.

### Proteolytic Treatment

The proteases, human MMP2, human MMP9, mouse MMP2 and mouse MMP9 were purchased from R&D systems. The protease digestion was carried out by incubating 10 μg-50 μg of prodrugs with 1 μg of human MMP2, human MMP9, mouse MMP2 or mouse MMP9 in the HBS buffer (20 mM HEPES, 150 mM NaCl₂, pH 7.4) containing 2 mM CaCl2 and 10 μM ZnCl₂ at 37° C. for 12 hours.

### Cell-Based Activity Assay

The prodrugs prior to the protease digestion and the control samples were tested by the cell-based activity assay. Briefly, NK92 cells were grown in the RPMI-1640 medium supplemented with L-glutamine, 10% fetal bovine serum, 10% non-essential amino acids, 10% sodium pyruvate, and 55 μM beta-mercaptoethanol. NK92 cells were non-adherent and maintained at 1×10⁵-1×10⁶ cells/ml in medium with 100 ng/ml of IL-2. Generally, cells were split twice per week. For bioassays, it was best to use cells no less than 48 hours after passage. IL-21 functional activity was determined by culturing NK92 cells at 5×10⁴ cells/well with serial dilutions of the samples in the presence of a constant amount of IL-2 for 2 days. Supernatants were then assayed for interferon-γ by ELISA.

### Mino IL-21 Viability Assay

The Mino cell viability assay is carried out following the protocol below:
a) Perform serial dilutions of test articles in 50 uL assay medium (RPMI 1640, 10% Fetal Bovine Serum, NEAA, sodium pyruvate, 55 μM b-mercaptoethanol) in 96 well tissue culture plate.
b) Add 20,000 Mino cells/well in 50 μL assay medium.
c) Culture for 3 or 5 days.
d) Add 100 μL/well Cell Titer Glo (Promega). Cell Titer-Glo provides a measure of cell viability by providing a quantitative assessment of ATP.
e) Measure luminescence.
   Ki67 Activation Assay with PBMC Primary Cells

Human PBMCs were stimulated with anti-CD3 antibody, OKT3 at 100 ng/mL for 2 days, followed by three-time washes, then were rested for 3 days in regular cell culture medium in 37° C., 5% CO₂ incubator. After that stimulated/rested PBMCs were seeded at 200K cells/well of 96-well plates and were treated with test articles as indicated at various concentrations for 5 days before subject to cell staining and flow cytometry analysis for Ki67 in PD-1⁺CD4⁺ and PD-1-CD4+ cell populations (all Abs used for cell staining were purchased from BD).

### Example 1: Construction of Anti-PD-1 Antibody-IL-21 Prodrug Fusion Molecules

An anti-PD-1 antibody-based IL-21 prodrug was constructed with two identical light chains (with an amino acid sequence as shown in SEQ ID NO: 44). A first heavy chain polypeptide chain (with an amino acid sequence as shown in SEQ ID NO: 42) and a second heavy chain polypeptide chain (with an amino acid sequence as shown SEQ ID NO: 76). The molecule was transiently expressed and purified (lot #PW04-38).

A second PD-1 antibody-based IL-21 prodrug with the scFv as the masking moiety was also expressed and purified (Lot #PW05-68).

In addition, as a control, the anti-PD-1 antibody-IL-21 fusion molecule without the mask was also expressed and purified (Lot #PW05-67).

Further, a second control, the anti-PD-1 antibody-IL-21 mutein (R9ER76A) fusion molecule without the mask was also expressed and purified (Lot #PW09-02).

### Example 2: Biological Activities of Anti-PD-1 Antibody-IL-21 Prodrug Fusion Molecules

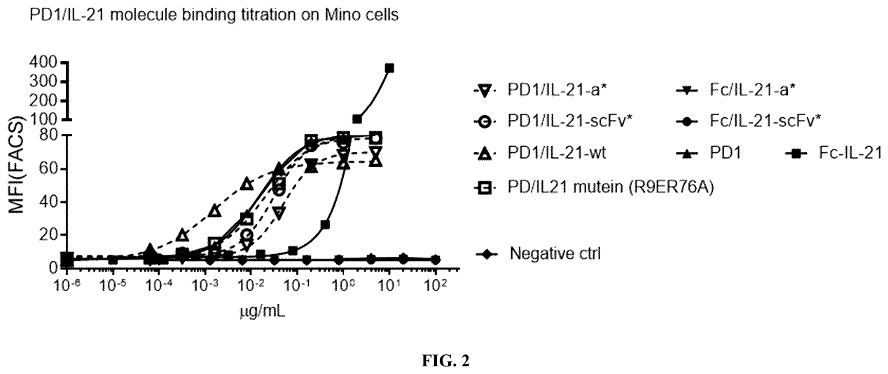

The binding of the prodrug molecules and several control molecules to the Mino cells were tested by FACS. The results on FIG. 2 show that both the PD-1 antibody and the Fc-IL-21 fusion molecule were able to bind to the Mino cells, indicating that the Mino cells express both the PD-1 and the receptors for IL-21. The results showed that both of the Fc-based IL-21 prodrug molecules had no binding to the cells, suggesting the IL-21 cytokine moieties have been masked by the corresponding masking moiety. However, the PD-1 antibody-based IL-21 prodrug molecules and fusion molecules were able to bind to the Mino cells.

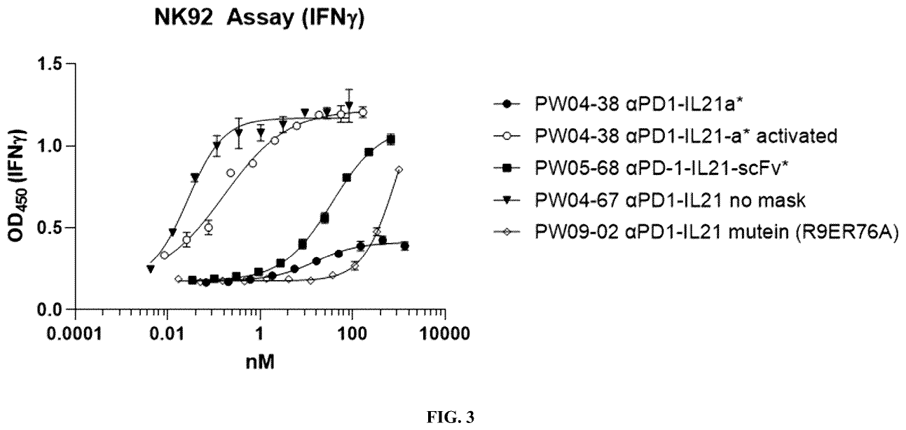

Cell-based activity of the molecules were assessed using NK92 cells, a natural killer (NK) cell line. The results are shown in FIG. 3. The data show that without activation, the prodrug molecule with the IL-21a ECD as the masking moiety (Lot #PW04-38) had minimum activity while the prodrug with an scFv as the masking moiety (Lot #PW05-68) had an activity ~1000 times lower than the one without the masking moiety (PW04-67). The data show that the bioassay activities of the prodrugs were significantly enhanced by the protease MMP2 treatment.

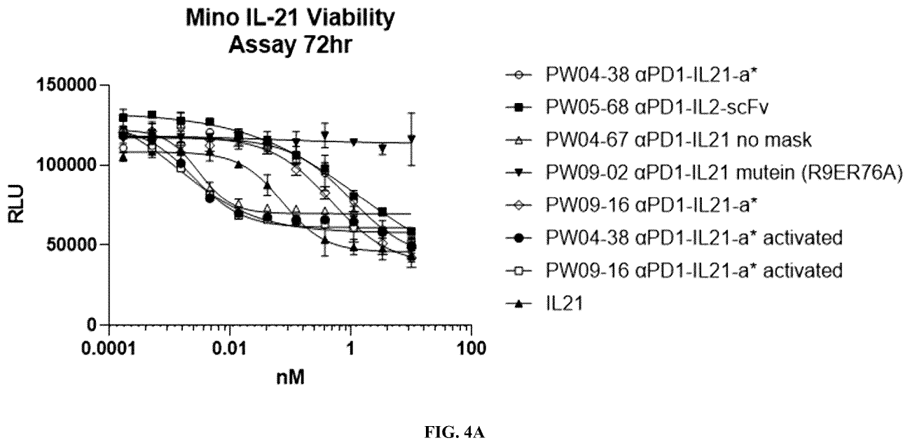

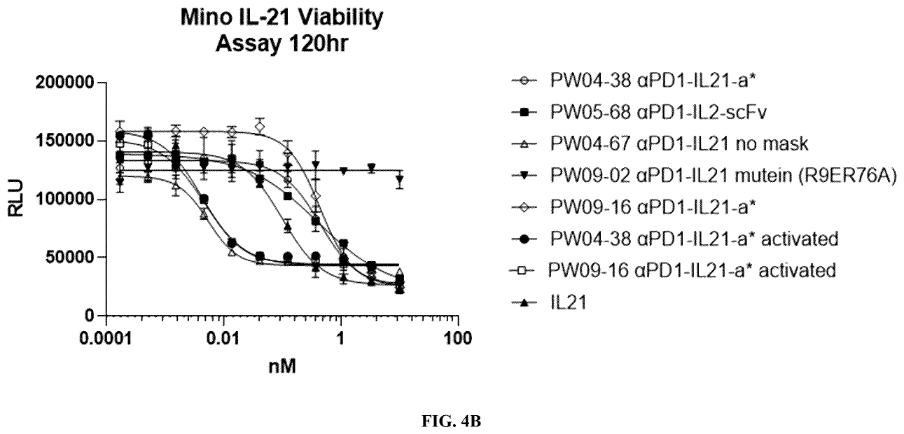

To test the tumor killing activity of the fusion molecules, Mino cells were used. Mino cells are a mantle cell lymphoma cell line that expresses both PD-1 and receptors for IL-21 (Harington et al., Leuk Lymphoma (2019) 60(10):2498-2507 and Gelebart et al., Leukemia (2009) 23:1836-1846). The Mino viability assay results are shown in FIGS. 4A and 4B. Surprisingly, the anti-PD-1 antibody-based IL-21 prodrugs (Lots #PW04-38 and PW05-68) had significant activities prior to activation, while the control molecule (PD-1 antibody-IL-21R9E/R76A fusion molecule, Lot #PW09-02) had no or little activity. The results demonstrate that the Anti-PD-1 IL-21 fusion molecule was activated through “cis-biding,” i.e., through binding to both the PD-1 and the IL-21 receptor(s). Cis-binding of the PD-1 antibody to the PD-1 antigen on the cell surface and the cytokine to its receptor on the same cell surface may have unraveled the masking effect of the masking moiety. It is therefore expected that prodrugs without cleavable peptide linker may be “activated” in a disease site such as a tumor because the local immune cells may express both the antigen targeted by the carrier and the receptor(s), which bind the cytokine moiety (e.g., IL-21).

### Example 3: Construction of Anti-PD-1 Antibody-IL-15 Prodrug Fusion Molecules

A series of anti-PD-1 antibody-based IL-15 prodrugs were constructed, where the IL-15 moiety (i) was masked with an scFv 215β, (ii) was masked an scFv 215γ, (iii) had no mask (215β Ref), or (iv) had mutations but no mask (PD1/IL15 mutein (M2)). The anti-PD-1 antibody in these prodrugs comprises two identical light chains with an amino acid sequence of SEQ ID NO: 44 and two identical heavy chains with an amino acid sequence of SEQ ID NO: 47.

The Fc-based prodrug 215β is a heterodimer comprising amino acid sequence ID NOs: 16 and 17.

The anti-PD-1 antibody-based prodrug 215γ comprises two identical light chains (SEQ ID NO: 44), a first heavy chain fusion polypeptide chain with an amino acid sequence of SEQ ID NO: 37 and a second heavy chain fusion polypeptide chain with an amino acid sequence of SEQ ID NO: 77.

215β Ref is an IL-15 mutein (D30N/E64Q/N65D)-Fc fusion molecule.

PD1/IL15 mutein (M2) is an anti-PD-1-antibody fused with an IL-15 mutein wherein the IL-15 agonist comprises mutations E46G, V49R, E64Q, D30N, and N1G.

All the samples were expressed by CHO cells and purified using a Protein A affinity chromatography followed by additional chromatography steps.

### Example 4: Biological Activities of Anti-PD-1 Antibody-IL-15 Prodrug Fusion Molecules

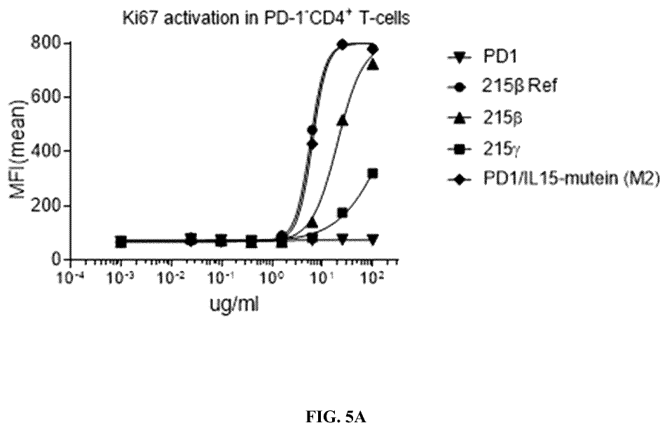

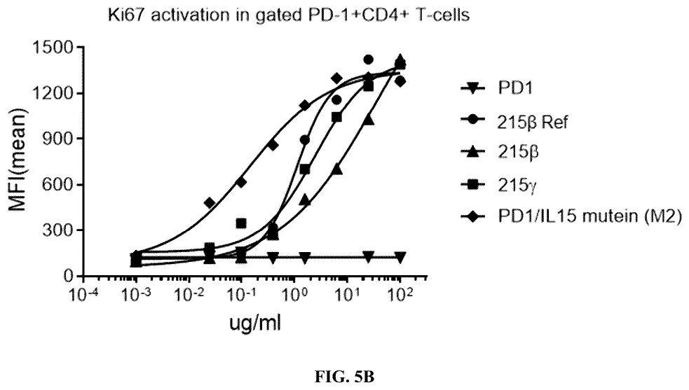

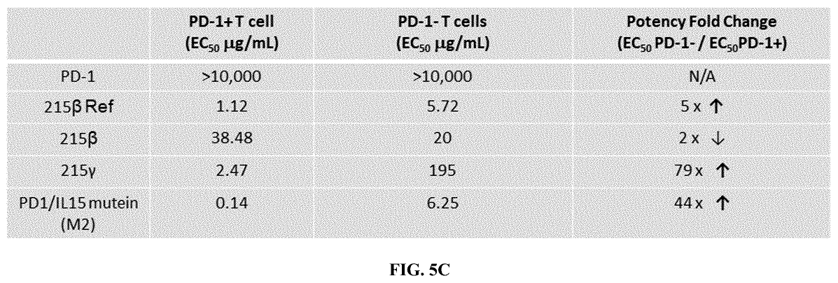

The IL-15 prodrugs were assessed for their ability to stimulate Ki67 expression in PBMC cells obtained from human donors. FIGS. 5A-5C show the results of Ki67 activation of the CD4⁺ T cells in PBMC after treatment. FIG. 5A shows the results of Ki67 activation of CD4+ T cells without PD-1 expression. FIG. 5B shows the results of Ki67 activation of CD4+ T cells with PD-1 expression. FIG. 5C shows the EC₅₀ values of the testing articles with CD4+ T cells without and with PD-1 expression. It also showed the fold of changes of the EC50 values between the CD4+ T cells without and with PD-1 expression. Surprisingly, the data showed that 215γ had significantly higher Ki67 activity with CD4⁺ cells expressing PD-1 than the ones without PD-1 expression, indicating that the prodrug 215γ was able to be activated without protease cleavage when it was acting with the cells expressing both the antigen PD-1 and the receptors for the cytokine IL-15.

### Example 5: Anti-PD-1 Antibody-IL-2 Prodrug Fusion Molecules

An anti-PD-1 antibody-based IL-2 prodrugs was similarly constructed. The prodrug, PD-1/IL-2v*, comprises two identical light chains (SEQ ID NO: 44) and two heavy chain fusion polypeptides (SEQ ID NOs: 24 and 25, respectively). The IL-2 moiety is an IL-2 mutein, and the mask in the prodrug is an IL-2 receptor β extracellular domain. The IL-2 prodrug was assessed for its ability to stimulate Ki67 expression in PBMC cells obtained from human donors.

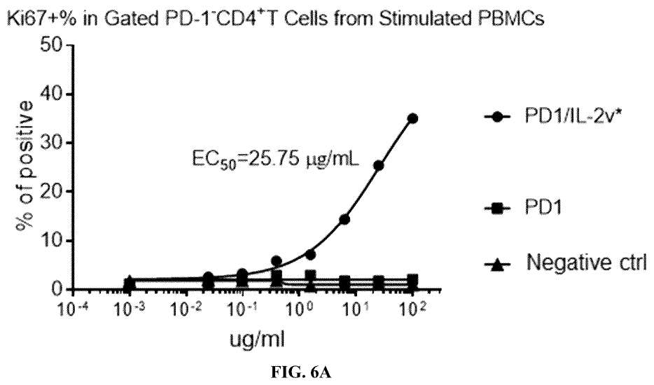

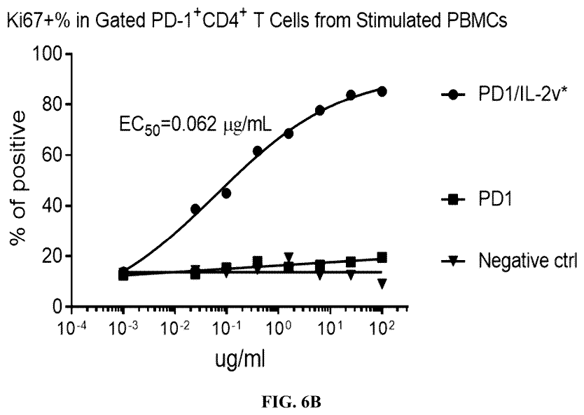

FIGS. 6A and 6B show the results of Ki67 activation in the CD4⁺ T cells from PBMC after treatment with an anti-PD-1 antibody (PD-1), a negative control, or PD-1/IL-2v*. The negative control was an IgG1 antibody against human Claudin 18.2. All the samples were expressed by CHO cells and purified using a Protein A affinity chromatography followed by additional chromatography steps. FIG. 6A shows the results of Ki67 activation in CD4⁺ T cells without PD-1 expression. FIG. 6B shows the results of Ki67 activation in CD4⁺ T cells with PD-1 expression. Surprisingly, the data showed that the prodrug PD-1/IL2V* had significantly higher Ki67-inducing activity on CD4+ cells expressing PD-1 than on those without PD-1 expression. These results indicate that PD-1/IL-2v* was able to be activated without protease cleavage when it was acting on cells expressing both the antigen PD-1 and the receptors for the cytokine IL-2.

Collectively the above data indicate that the cytokine prodrugs were able to be activated at a site where there were cells were expressing both the receptors for the cytokines and the antigens targeted by the carrier moiety. It was surprising that the prodrugs with carriers having antigen-binding moieties were able to be activated without protease cleavage. While not wishing to be bound by theory, we expect that the antigen-binding domain of the carrier moiety targets the prodrug molecules to the surface of the cells expressing the antigens and this allows the cytokine receptors on the cell surface to compete effectively with the mask moiety of the prodrug. This process culminates in protease cleavage-free activation of the prodrug via displacement of the masking moiety the cytokine receptors on the cell surface, as illustrated in FIG. 7.

Cells like exhausted Teff cells express both the antigen (e.g., PD-1) and the receptors for the cytokine (e.g., IL-2Rβ and IL-2Rγ). Exhausted Teff cells are in general localized at tumor microenvironment but in the normal tissues. This differential distribution of the cells expressing the antigens and the cytokine receptors provides the disease site-specific activation of the prodrugs. Such target specificity would also result in enhanced safety for the cytokine therapeutics.

SEQUENCES

SEQ ID NO: 1 - human IL-21
QGQDRHMIRM RQLIDIVDQL KNYVNDLVPE FLPAPEDVET NCEWSAFSCF QKAQLKSANT
GNNERIINVS IKKLKRKPPS TNAGRRQKHR LTCPSCDSYE KKPPKEFLER FKSLLQKMIH
QHLSSRTHGS EDS
SEQ ID NO: 2 - IL-21 Mutein A
QGQDRHMIRM RQLIDIVAQL KNYVNDLVPE FLPAPEDVET NCEWSAFSCF QKAQLKSANT
GNNERIINVS IKKLKRKPPS TNAGRRQKHR LTCPSCDSYE KKPPKEFLER FKSLLQKMIH
QHLSSRTHGS EDS
SEQ ID NO: 3 - IL-21 Mutein B
QGQDRHMIRM RQLIDIVKQL KNYVNDLVPE FLPAPEDVET NCEWSAFSCF QKAQLKSANT
GNNERIINVS IKKLKRKPPS TNAGRRQKHR LTCPSCDSYE KKPPKEFLER FKSLLQKMIH
QHLSSRTHGS EDS
SEQ ID NO: 4 - IL-21 Mutein C
QGQDRHMIRM RQLIDIVDQL KNYVNDLVPE FLPAPEDVET NCEWSAFSCF QKAQLKSANT
GNNERIINVS IKKLKRKPPS TNAGRRQKHR LTCPSCDSYE KKPPKEFLRR FKSLLQKMIH
QHLSSRTHGS EDS
SEQ ID NO: 5 - IL-21 Mutein D
QGQDRHMIRM RQLIDIVDKL KNYVNDLVPE FLPAPEDVET NCEWSAFSCF QKAQLKSANT
GNNERIINVS IKKLKRKPPS TNAGRRQKHR LTCPSCDSYE KKPPKEFLRR FKSLLQKMIH
QHLSSRTHGS EDS
SEQ ID NO: 6 - Human IL-2
APTSSSTKKT QLQLEHLLLD LQMILNGINN YKNPKLTRML TFKFYMPKKA TELKHLQCLE
EELKPLEEVL NLAQSKNFHL RPRDLISNIN VIVLELKGSE TTFMCEYADE TATIVEFLNR
WITFCQSIIS TLT
SEQ ID NO: 7 - Human IL-15
NWVNVISDLK KIEDLIQSMH IDATLYTESD VHPSCKVTAM KCFLLELQVI SLESGDASIH
DTVENLIILA NNSLSSNGNV TESGCKECEE LEEKNIKEFL QSFVHIVQMF INT
SEQ ID NO: 8 - Human IL-15 Rα sushi domain
ITCPPPMSVE HADIWVKSYS LYSRERYICN SGFKRKAGTS SLTECVLNKA TNVAHWTTPS
LKCIRDPALV HQRPA
SEQ ID NO: 9 - Human IL-7
DCDIEGKDGK QYESVLMVSI DQLLDSMKEI GSNCLNNEFN FFKRHICDAN KEGMFLFRAA
RKLRQFLKMN STGDFDLHLL KVSEGTTILL NCTGQVKGRK PAALGEAQPT KSLEENKSLK
EQKKLNDLCF LKRLLQEIKT CWNKILMGTK EH
SEQ ID NO: 10 - Human IL-9
QGCPTLAGIL DINFLINKMQ EDPASKCHCS ANVTSCLCLG IPSDNCTRPC FSERLSQMTN
TTMQTRYPLI FSRVKKSVEV LKNNKCPYFS CEQPCNQTTA GNALTFLKSL LEIFQKEKMR
GMRGKI
SEQ ID NO: 11 - IL-2Rβ ECD
AVNGTSQFTC FYNSRANISC VWSQDGALQD TSCQVHAWPD RRRWNQTCEL LPVSQASWAC
NLILGAPDSQ KLTTVDIVTL RVLCREGVRW RVMAIQDFKP FENLRLMAPI SLQVVHVETH
RCNISWEISQ ASHYFERHLE FEARTLSPGH TWEEAPLLTL KQKQEWICLE TLTPDTQYEF
QVRVKPLQGE FTTWSPWSQP LAFRTKPAAL GKDT
SEQ ID NO: 12 - IL-21Rα ECD (source: uniprot.org/uniprot/Q9HBE5)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SDYEDPAFYM LKGKLQYELQ YRNRGDPWAV SPRRKLISVD SRSVSLLPLE FRKDSSYELQ
VRAGPMPGSS YQGTWSEWSD PVIFQTQSEE LKE
SEQ ID NO: 13 - Human IL-21Rγ ECD
LNTTILTPNG NEDTTADFFL TTMPTDSLSV STLPLPEVQC FVFNVEYMNC TWNSSSEPQP
TNLTLHYWYK NSDNDKVQKC SHYLFSEEIT SGCQLQKKEI HLYQTFVVQL QDPREPRRQA
TQMLKLQNLV IPWAPENLTL HKLSESQLEL NWNNRFLNHC LEHLVQYRTD WDHSWTEQSV
DYRHKFSLPS VDGQKRYTFR VRSRFNPLCG SAQHWSEWSH PIHWGSNTSK ENPFLFALEA
SEQ ID NO: 14 - IL-7α receptor ECD
(Source: https://www.uniprot.org/uniprot/P16871)
ESGYAQNGDL EDAELDDYSF SCYSQLEVNG SQHSLTCAFE DPDVNITNLE FEICGALVEV
KCLNFRKLQE IYFIETKKFL LIGKSNICVK VGEKSLTCKK IDLTTIVKPE APFDLSVVYR
EGANDFVVTF NTSHLQKKYV KVLMHDVAYR QEKDENKWTH VNLSSTKLTL LQRKLQPAAM
YEIKVRSIPD HYFKGFWSEW SPSYYFRTPE INNSSGEMD
SEQ ID NO: 15 - IL-9α receptor ECD (source:)
SVTGEGQGPR SRTFTCLTNN ILRIDCHWSA PELGQGSSPW LLFTSNQAPG GTHKCILRGS
ECTVVLPPEA VLVPSDNFTI TFHHCMSGRE QVSLVDPEYL PRRHVKLDPP SDLQSNISSG
HCILTWSISP ALEPMTTLLS YELAFKKQEE AWEQAQHRDH IVGVTWLILE AFELDPGFIH
EARLRVQMAT LEDDVVEEER YTGQWSEWSQ PVCFQAPQRQ GPLIPPWGWP
SEQ ID NO: 16 Fc-scFv2 against IL-15
DKTHTCPPCP APEAAGGPSV FLFPPKPKDT LYITREPEVT CVVVDVSHED PEVKFNWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVFSCSVMHE ALHNHYTQKS LSLSPGAGGG GSVHMPLGFL
GPRQARVVNG GGGGSGGGGS EIVLTQSPGT LSLSPGERAT LSCRASQSVS SSYLAWYQQK
PGQAPRLLIY GASRRATGIP DRFSGSGSGT DFTLTISRLE PEDFAVYYCQ RYGSSHTFGQ
GTKLEISGGG GSGGGGSGGG GSEVQLVQSG AEVKKPGESL KISCKVSGYF FTTYWIGWVR
QMPGKGLEYM GIIYPGDSDT RYSPSFQGQV TISADKSIST AYLQWSSLKA SDTAMYYCAR
GGNWNCFDYW GQGTLVTVSS
SEQ ID NO: 17 Fc-IL-15 N65D
DKTHTCPPCP APEAAGGPSV FLFPPKPKDT LYITREPEVT CVVVDVSHED PEVKFNWYVD
GVEVHNAKTK PREEQYNSTY RVVSVLTVLH QDWLNGKEYK CKVSNKALPA PIEKTISKAK
GQPREPQVYT LPPCREEMTK NQVSLWCLVK GFYPSDIAVE WESNGQPENN YKTTPPVLDS
DGSFFLYSKL TVDKSRWQQG NVFSCSVMHE ALHNHYTQKS LSLSPGAGGG GSGGGGSGGG
GSITCPPPMS VEHADIWVKS YSLYSRERYI CNSGFKRKAG TSSLTECVLN KATNVAHWTT
PSLKCIRGGG GSGGGSGGGG SAAGGGGSGG GGSGGGGSNW VNVISDLKKI EDLIQSMHID
ATLYTESDVH PSCKVTAMKC FLLELQVISL ESGDASIHDT VEDLIILANN SLSSNGNVTE
SGCKECEELE EKNIKEFLQS FVHIVQMFIN TS
SEQ ID NO: 18 - Anti-IL-15 scFv1 (italic: VH and VL)
EVQLVQSGAE VKKPGESLKI SCKVSGYFFT TYWIGWVRQM PGKGLEYMGI IYPGDSDTRY
SPSFQGQVTI SADKSISTAY LQWSSLKASD TAMYYCARGG NWNCFDYWGQ GTLVTVSSGG
GGSGGGGSGG GGSGIVLTQS PGTLSLSPGE RATLSCRASQ SVSSSYLAWY QQKPGQAPRL
LIYGASRRAT GIPDRFSGSG SGTDFTLTIS RLEPEDFAVY YCQRYGSSHT FGQGTKLEIS
SEQ ID NO: 19 - Anti-IL-15 scFv2 (italic: VL and VH)
EIVLTQSPGT LSLSPGERAT LSCRASQSVS SSYLAWYQQK PGQAPRLLIY GASRRATGIP
DRFSGSGSGT DFTLTISRLE PEDFAVYYCQ RYGSSHTFGQ GTKLEISGGG GSGGGGSGGG
GSEVQLVQSG AEVKKPGESL KISCKVSGYF FTTYWIGWVR QMPGKGLEYM GIIYPGDSDT
RYSPSFQGQV TISADKSIST AYLQWSSLKA SDTAMYYCAR GGNWNCFDYW GQGTLVTVSS
SEQ ID NO: 20 - Anti-IL-21 scFv1 (italic: VH and VL)
QVQLVESGGG WQPGRSLRL SCAASGFTFS SYGMHWVRQA PGKGLEWVAF IWYDGSDKYY
ADSVKGRFTI SRDNSKNTLY LQMNSLRAED TAVYYCARDG DSSDWYGDYY FGMDVWGQGT
TVTVSSGGGG SGGGGSGGGG SEIVLTQSPG TLSLSPGERA TLSCRASQSV SSSYLAWYQQ
KPGQAPRLLI YGASSRATGI PDRFSGSGSG TDFTLTISRL EPEDFAVYYC QQYGSWTFGQ
GTKVEIK
SEQ ID NO: 21 - Anti-IL-21 scFv2 (italic: VL and VH)
EIVLTQSPGT LSLSPGERAT LSCRASQSVS SSYLAWYQQK PGQAPRLLIY GASSRATGIP
DRFSGSGSGT DFTLTISRLE PEDFAVYYCQ QYGSWTFGQG TKVEIKGGGG SGGGGSGGGG
SQVQLVESGG GVVQPGRSLR LSCAASGFTF SSYGMHWVRQ APGKGLEWVA FIWYDGSDKY
YADSVKGRFT ISRDNSKNTL YLQMNSLRAE DTAVYYCARD GDSSDWYGDY YFGMDVWGQG
TTVTVSS
SEQ ID NO: 22 - Anti-IL-2 scFv1 (underlined: HCDR1-3 and LCDR1-3;
italic: VH and VL)
QVQLVQSGAE VKKPGSSVKV SCKASGGTFS SYAISWVRQA PGQGLEWMGG IIPIFGTANY
AQKFQGRVTI TADESTSTAY MELSSLRSED TAVYYCARVD RYYNWNYFLG SFDYWGQGTL
VTVSSGGGGS GGGGSGGGGS SYVLTQPPSV SVAPGKTARI TCGGNNIRSK SVHWYQQKPG
QAPVVVIYYD SDRPSGIPER ISGSNSGNTA TLTISRVEAG DEADYFCQVW DSSSDHHVFG
GGTKLTVL
SEQ ID NO: 23 - Anti-IL-2 scFv2 (underlined: LCDR1-3 and HCDR1-3;
italic: VL and VH)
SYVLTQPPSV SVAPGKTARI TCGGNNIRSK SVHWYQQKPG QAPWVIYYD SDRPSGIPER
ISGSNSGNTA TLTISRVEAG DEADYFCQVW DSSSDHHVFG GGTKLTVLGG GGSGGGGSGGG
GSQVQLVQSG AEVKKPGSSV KVSCKASGGT FSSYAISWVR QAPGQGLEWM GGIIPIFGTA
NYAQKFQGRV TITADESTST AYMELSSLRS EDTAVYYCAR VDRYYNWNYF LGSFDYWGQG
TLVTVSS
SEQ ID NO: 24 - PD1-HC-IL-2V (CX3.58.1)
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPCQEE MTKNQVSLWC
LVKGFYPSDI AVEWESNGQP ENNYKTTPPV LDSDGSFFLY SRLTVDKSRW QEGNVFSCSV
MHEALHNHYT QKSLSLSLGK GGGGSGGGGS GGGGSAPASS STKKTQLQLE HLLLDLQMIL
NGINNYKNPK LTSMLTAKFA MPKKATELKH LQCLEEALKP LEEVLNLAQS KNFHLRPRDL
ISNINVIVLE LKGSETTFMC EYADETATIV EFLNRWITFS QSIISTLT
SEQ ID NO: 25 - PD1-HC-IL-2Rβ-ECD (CX3.58.1)
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VCTLPPSQEE MTKNQVSLSC
AVKGFYPSDI AVEWESNGQP ENNYKTTPPV LDSDGSFFLV SRLTVDKSRW QEGNVFSCSV
MHEALHNHYT QKSLSLSLGK GGGGSGPLGV RGGGGSGGGG SAVNGTSQFT CFYNSRANIS
CVWSQDGALQ DTSCQVHAWP DRRRWNQTCE LLPVSQASWA CNLILGAPDS QKLTTVDIVT
LRVLCREGVR WRVMAIQDFK PFENLRLMAP ISLQVVHVET HRCNISWEIS QASHYFERHL
EFEARTLSPG HTWEEAPLLT LKQKQEWICL ETLTPDTQYE FQVRVKPLQG EFTTWSPWSQ
PLAFRTKPAA LGKDT
SEQ ID NO: 26 - PD1-HC-IL-21Rα ECD WITH NON-CLEAVABLE PEPTIDE LINKER
MGVKVLFALI CIAVAEAQVQ LVESGGGVVQ PGRSLRLDCK ASGITFSNSG MHWVRQAPGK
GLEWVAVIWY DGSKRYYADS VKGRFTISRD NSKNTLFLQM NSLRAEDTAV YYCATNDDYW
GQGTLVTVSS ASTKGPSVFP LAPCSRSTSE STAALGCLVK DYFPEPVTVS WNSGALTSGV
HTFPAVLQSS GLYSLSSVVT VPSSSLGTKT YTCNVDHKPS NTKVDKRVES KYGPPCPPCP
APEFLGGPSV FLFPPKPKDT LMISRTPEVT CVVVDVSQED PEVQFNWYVD GVEVHNAKTK
PREEQFNSTY RVVSVLTVLH QDWLNGKEYK CKVSNKGLPS SIEKTISKAK GQPREPQVYT
LPPSQEEMTK NQVSLTCLVK GFYPSDIAVE WESNGQPENN YKTTPPVLDS DGSFFLYSRL
TVDKSRWQEG NVFSCSVMHE ALHNHYTQKS LSLSLGAGGG GSGGGGSAAG GGGSGGGGSC
PDLVCYTDYL QTVICILEMW NLHPSTLTLT WQDQYEELKD EATSCSLHRS AHNATHATYT
CHMDVFHFMA DDIFSVNITD QSGNYSQECG SFLLAESIKP APPFNVTVTF SGQYNISWRS
DYEDPAFYML KGKLQYELQY RNRGDPWAVS PRRKLISVDS RSVSLLPLEF RKDSSYELQV
RAGPMPGSSY QGTWSEWSDP VIFQTQSEEL KE
SEQ ID NO: 27-34 Peptide Linker
(SEQ ID NO: 27)
(GGGGS)ₙ,
wherein n = 1, 2, 3, and 4
(SEQ ID NO: 28)
GGGGS
(SEQ ID NO: 29)
GGGGSGGGGS
(SEQ ID NO: 30)
GGGGSGGGGSGGGGS
(SEQ ID NO: 31)
GGGGSGGGGSXXGGGGSGGGGS,
X = A or N
(SEQ ID NO: 32)
GGGGSGGGGSGGGGSXXGGGGSGGGGS,
X = A or N
(SEQ ID NO: 33)
GGGGSGGGGSGGGGSXXGGGGSGGGGSGGGGS,
X = A or N
(SEQ ID NO: 34)
GGGGSGGGGXGGGGYGGGGS,
X = S, A or N, and Y = A or N
SEQ ID NO: 35 - PD1-HC-IL-2V
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLA (GGGGS)ₙ₁X₁X₂(GGGGS)ₙ₂APXₐₐ₃SSSTKKTQL QLEHLLLDLQ
MILNGINNYK NPXₐₐ₃₅LTRMLTXₐₐ₄₂KFXₐₐ₄₅M PKKATELKH LQCLEEELKP
LEEVLNXₐₐ₇₂Xₐₐ₇₃QSKNFHLR PRDLISNINV IVLELKGSET TFMCEYADET ATIVEFLNRW
ITFXₐₐ₁₂₅QSIIS TLT
wherein Xₐₐ₃ is N or A;
wherein Xₐₐ₁₂₅ is A or S;
wherein Xₐₐ₃₅ is selected from K and N;
wherein Xₐₐ₄₂ is selected from A, G, S, T, Q, E, N, D, R, and K;
wherein Xₐₐ₄₅ is selected from A, G, S, T, Q, E, N, D, R, and K;
wherein Xₐₐ₇₂ is selected from A, G, S, T, Q, E, N, D, R, and K;
wherein Xₐₐ₇₃ is selected from A and T;
wherein n₁ = 0, 1, 2, or 3;
n₂ = 0, 1, 2, or 3; and
X₁ is an amino acid Alanine or deleted; and
X₂ is an amino acid Alanine or deleted.
SEQ ID NO: 36 - PD1-HC-IL-2Rβ-ECD
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGA(GGGGS)ₙ₁X₁X₂(GGGGS)n₂AV NGTSQFTCFY NSRANISCVW
SQDGALQDTS CQVHAWPDRR RWNQTCELLP VSQASWACNL ILGAPDSQKL TTVDIVTLRV
LCREGVRWRV MAIQDFKPFE NLRLMAPISL QVVHVETHRC NISWEISQAS HYFERHLEFE
ARTLSPGHTW EEAPLLTLKQ KQEWICLETL TPDTQYEFQV RVKPLQGEFT TWSPWSQPLA
FRTKPAALGK DT
Wherein n₁ = 0, 1, 2, or 3; n₂ = 0, 1, 2, or 3; X₁ is an amino acid
Alanine or deleted; and X₂ is an amino acid Alanine or deleted.
SEQ ID NO: 37 - PD1-HC with-SUSHI-IL-15V
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGA GGGGSGGGGS GGGGSITCPP PMSVEHADIW VKSYSLYSRE
RYICNSGFKR KAGTSSLTEC VLNKATNVAH WTTPSLKCIR GGGGSGGGSG GGGSAAGGGG
SGGGGSGGGG SNWVNVISDL KKIEDLIQSM HIDATLYTES DVHPSCKVTA MKCFLLELQV
ISLESGDASI HDTVEDLIIL ANNSLSSNGN VTESGCKECE ELEEKNIKEF LQSFVHIVQM
PINTS
SEQ ID NO: 38 - PD1-HC-SCFV2 AGAINST IL-15
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGA(GGGGS)ₙ₁X₁X₂(GGGGS)ₙ₂EIVLTQSPGTLS LSPGERATLS
CRASQSVSSS YLAWYQQKPG QAPRLLIYGA SRRATGIPDR FSGSGSGTDF TLTISRLEPE
DFAVYYCQRY GSSHTFGQGT KLEISGGGGS GGGGSGGGGS EVQLVQSGAE VKKPGESLKI
SCKVSGYFFT TYWIGWVRQM PGKGLEYMGI IYPGDSDTRY SPSFQGQVTI SADKSISTAY
LQWSSLKASD TAMYYCARGG NWNCFDYWGQ GTLVTVSS
Wherein n₁ = 0, 1, 2, or 3; n₂ = 0, 1, 2, or 3; X₁ is an amino acid
Alanine or deleted; and X₂ is an amino acid Alanine or deleted.
SEQ ID NO: 39 - PD1-HC-IL-21
MGVKVLFALI CIAVAEAQVQ LVESGGGVVQ PGRSLRLDCK ASGITFSNSG MHWVRQAPGK
GLEWVAVIWY DGSKRYYADS VKGRFTISRD NSKNTLFLQM NSLRAEDTAV YYCATNDDYW
GQGTLVTVSS ASTKGPSVFP LAPCSRSTSE STAALGCLVK DYFPEPVTVS WNSGALTSGV
HTFPAVLQSS GLYSLSSVVT VPSSSLGTKT YTCNVDHKPS NTKVDKRVES KYGPPCPPCP
APEFLGGPSV FLFPPKPKDT LMISRTPEVT CVVVDVSQED PEVQFNWYVD GVEVHNAKTK
PREEQFNSTY RVVSVLTVLH QDWLNGKEYK CKVSNKGLPS SIEKTISKAK GQPREPQVYT
LPPSQEEMTK NQVSLTCLVK GFYPSDIAVE WESNGQPENN YKTTPPVLDS DGSFFLYSRL
TVDKSRWQEG NVFSCSVMHE ALHNHYTQKS LSLSLGAGGG GSGGGGSGGG GSQGQDRHMI
RMRQLIDIVD QLKNYVNDLV PEFLPAPEDV ETNCEWSAFS CFQKAQLKSA NTGNNERIIN
VSIKKLKRKP PSTNAGRRQK HRLTCPSCDS YEKKPPKEFL ERFKSLLQKM IHQHLSSRTH
GSEDS
SEQ ID NO: 40 - PD1-HC-IL-21Rα ECD WITH CLEAVABLE PEPTIDE LINKER
MGVKVLFALI CIAVAEAQVQ LVESGGGVVQ PGRSLRLDCK ASGITFSNSG MHWVRQAPGK
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGA GGGGSGGGGS GPLGVRGGGG SCPDLVCYTD YLQTVICILE
MWNLHPSTLT LTWQDQYEEL KDEATSCSLH RSAHNATHAT YTCHMDVFHF MADDIFSVNI
TDQSGNYSQE CGSFLLAESI KPAPPFNVTV TFSGQYNISW RSDYEDPAFY MLKGKLQYEL
QYRNRGDPWA VSPRRKLISV DSRSVSLLPL EFRKDSSYEL QVRAGPMPGS SYQGTWSEWS
DPVIFQTQSE ELKE
SEQ ID NO: 41 - PD1-HC-SCFV2 AGAINST IL-21
MGVKVLFALI CIAVAEAQVQ LVESGGGVV QPGRSLRLDC KASGITFSNS GMHWVRQAPG
KGLEWVAVIW YDGSKRYYAD SVKGRFTIS RDNSKNTLFL QMNSLRAEDT AVYYCATNDD
YWGQGTLVTV SSASTKGPSV FPLAPCSRS TSESTAALGC LVKDYFPEPV TVSWNSGALT
SGVHTFPAVL QSSGLYSLSS VVTVPSSSL GTKTYTCNVD HKPSNTKVDK RVESKYGPPC
PPCPAPEFLG GPSVFLFPPK PKDTLMISR TPEVTCVVVD VSQEDPEVQF NWYVDGVEVH
NAKTKPREEQ FNSTYRVVSV LTVLHQDWL NGKEYKCKVS NKGLPSSIEK TISKAKGQPR
EPQVYTLPPS QEEMTKNQVS LTCLVKGFY PSDIAVEWES NGQPENNYKT TPPVLDSDGS
FFLYSRLTVD KSRWQEGNVF SCSVMHEAL HNHYTQKSLS LSLGAGGGGS GGGGSAAGGG
GSGGGGSEIV LTQSPGTLSL SPGERATLS CRASQSVSSS YLAWYQQKPG QAPRLLIYGA
SSRATGIPDR FSGSGSGTDF TLTISRLEP EDFAVYYCQQ YGSWTFGQGT KVEIKGGGGS
GGGGSGGGGS QVQLVESGGG VVQPGRSLR LSCAASGFTF SSYGMHWVRQ APGKGLEWVA
FIWYDGSDKY YADSVKGRFT ISRDNSKNT LYLQMNSLRA EDTAVYYCAR DGDSSDWYGD
YYFGMDVWGQ GTTVTVSS
SEQ ID NO: 42 - PD1-HC with knob mutations-IL-21
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPCQEE MTKNQVSLWC
LVKGFYPSDI AVEWESNGQP ENNYKTTPPV LDSDGSFFLY SRLTVDKSRW QEGNVFSCSV
MHEALHNHYT QKSLSLSLGA GGGGSGGGGS GGGGSQGQDR HMIRMRQLID IVDQLKNYVN
DLVPEFLPAP EDVETNCEWS AFSCFQKAQL KSANTGNNER IINVSIKKLK RKPPSTNAGR
RQKHRLTCPS CDSYEKKPPK EFLERFKSLL QKMIHQHLSS RTHGSEDS
SEQ ID NO: 43 - PD1-HC with hole mutations-IL-21Rα ECD
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VCTLPPSQEE MTKNQVSLSC
AVKGFYPSDI AVEWESNGQP ENNYKTTPPV LDSDGSFFLV SRLTVDKSRW QEGNVFSCSV
MHEALHNHYT QKSLSLSLGA GGGGSGGGGS AAGGGGSGGG GSCPDLVCYT DYLQTVICIL
EMWNLHPSTL TLTWQDQYEE LKDEATSCSL HRSAHNATHA TYTCHMDVFH FMADDIFSVN
ITDQSGNYSQ ECGSFLLAES IKPAPPFNVT VTFSGQYNIS WRSDYEDPAF YMLKGKLQYE
LQYRNRGDPW AVSPRRKLIS VDSRSVSLLP LEFRKDSSYE LQVRAGPMPG SSYQGTWSEW
SDPVIFQTQS EELKE
SEQ ID NO: 44 - PD1-LC
EIVLTQSPAT LSLSPGERAT LSCRASQSVS SYLAWYQQKP GQAPRLLIYD ASNRATGIPA
RFSGSGSGTD FTLTISSLEP EDFAVYYCQQ SSNWPRTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
SEQ ID NO: 45 - Anti-PDL1 atezolizumab LC
DIQMTQSPSS LSASVGDRVT ITCRASQDVS TAVAWYQQKP GKAPKLLIYS ASFLYSGVPS
RFSGSGSGTD FTLTISSLQP EDFATYYCQQ YLYHPATFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
SEQ ID NO: 46 - Anti-PDL1 atezolizumab HC
EVQLVESGGG LVQPGGSLRL SCAASGFTFS DSWIHWVRQA PGKGLEWVAW ISPYGGSTYY
ADSVKGRFTI SADTSKNTAY LQMNSLRAED TAVYYCARRH WPGGFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKKVEPKS CDKTHTCPPC PAPELLGGPS
VFLFPPKPKD TLMISRTPEV TCVVVDVSHE DPEVKFNWYV DGVEVHNAKT KPREEQYAST
YRVVSVLTVL HQDWLNGKEY KCKVSNKALP APIEKTISKA KGQPREPQVY TLPPSREEMT
KNQVSLTCLV KGFYPSDIAV EWESNGQPEN NYKTTPPVLD SDGSFFLYSK LTVDKSRWQQ
GNVFSCSVMH EALHNHYTQK SLSLSPGK
SEQ ID NO: 47 - Anti-PD-1 Nivolumab HC
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGK
SEQ ID NO: 48 - Anti-PD-1 Pembrolizumab LC
EIVLTQSPAT LSLSPGERAT LSCRASKGVS TSGYSYLHWY QQKPGQAPRL LIYLASYLES
GVPARFSGSG SGTDFTLTIS SLEPEDFAVY YCQHSRDLPL TFGGGTKVEI KRTVAAPSVF
IFPPSDEQLK SGTASVVCLL NNFYPREAKV QWKVDNALQS GNSQESVTEQ DSKDSTYSLS
STLTLSKADY EKHKVYACEV THQGLSSPVT KSFNRGEC
SEQ ID NO: 49 - Anti-PD-1 Pembrolizumab HC
QVQLVQSGVE VKKPGASVKV SCKASGYTFT NYYMYWVRQA PGQGLEWMGG INPSNGGTNF
QVQLVQSGVE VKKPGASVKV SCKASGYTFT NYYMYWVRQA YRFDMGFDYW GQGTTVTVSS
NEKFKNRVTL TTDSSTTTAY MELKSLQFDD TAVYYCARRD WNSGALTSGV HTFPAVLQSS
ASTKGPSVFP LAPCSRSTSE STAALGCLVK DYFPEPVTVS KYGPPCPPCP APEFLGGPSV
GLYSLSSVVT VPSSSLGTKT YTCNVDHKPS NTKVDKRVES GVEVHNAKTK PREEQFNSTY
FLFPPKPKDT LMISRTPEVT CVVVDVSQED PEVQFNWYVD GQPREPQVYT LPPSQEEMTK
RVVSVLTVLH QDWLNGKEYK CKVSNKGLPS SIEKTISKAK DGSFFLYSRL TVDKSRWQEG
NQVSLTCLVK GFYPSDIAVE WESNGQPENN YKTTPPVLDS DGSFFLYSRL TVDKSRWQEG
NVFSCSVMHE ALHNHYTQKS LSLSLGK
SEQ ID NO: 50 - CTLA-4 antibody Ipilimumab LC
EIVLTQSPGT LSLSPGERAT LSCRASQSVG SSYLAWYQQK PGQAPRLLIY GAFSRATGIP
DRFSGSGSGT DFTLTISRLE PEDFAVYYCQ QYGSSPWTFG QGTKVEIKRT VAAPSVFIFP
PSDEQLKSGT ASVVCLLNNF YPREAKVQWK VDNALQSGNS QESVTEQDSK DSTYSLSSTL
TLSKADYEKH KVYACEVTHQ GLSSPVTKSF NRGEC
SEQ ID NO: 51 - CTLA-4 antibody Ipilimumab HC-IL2v
QVQLVESGGG VVQPGRSLRL SCAASGFTFS SYTMHWVRQA PGKGLEWVTF ISYDGNNKYY
ADSVKGRFTI SRDNSKNTLY LQMNSLRAED TAIYYCARTG WLGPFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSRDELT
KNQVSLTCLV KGFYPSDIAV EWESNGQPEN NYKTTPPVLD SDGSFFLYSK LTVDKSRWQQ
GNVFSCSVMH EALHNHYTQK SLSLSPGA(GGGGS)ₙ₁X₁X₂(GGGGS)ₙ₂APXₐₐ₃ SSSTKKTQLQ
LEHLLLDLQM ILNGINNYKN PXₐₐ₃₅LTRMLTXₐₐ₄₂KFXₐₐ₄₅M PKKATELKHLQ CLEEELKPLE
EVLNXₐₐ₇₂Xₐₐ₇₃QSKNFHLRPR DLISNINVIV LELKGSETTF MCEYADETATI VEFLNRWITF
Xₐₐ₁₂₅QSIIST LT
wherein Xₐₐ₃ is N or A;
wherein Xₐₐ₁₂₅ is A or S;
wherein Xₐₐ₃₅ is selected from K and N;
wherein Xₐₐ₄₂ is selected from A, G, S, T, Q, E, N, D, R, and K;
wherein Xₐₐ₄₅ is selected from A, G, S, T, Q, E, N, D, R, and K;
wherein Xₐₐ₇₂ is selected from A, G, S, T, Q, E, N, D, R, and K;
wherein Xₐₐ₇₃ is selected from A and T;
wherein n₁ = 0, 1, 2, or 3;
n₂ = 0, 1, 2, or 3; and
X₁ is an amino acid Alanine or deleted; and
X₂ is an amino acid Alanine or deleted.
SEQ ID NO: 52 - CTLA-4 antibody Ipilimumab HC-IL15v
QVQLVESGGG VVQPGRSLRL SCAASGFTFS SYTMHWVRQA PGKGLEWVTF ISYDGNNKYY
ADSVKGRFTI SRDNSKNTLY LQMNSLRAED TAIYYCARTG WLGPFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSRDELT
KNQVSLTCLV KGFYPSDIAV EWESNGQPEN NYKTTPPVLD SDGSFFLYSK LTVDKSRWQQ
GNVFSCSVMH EALHNHYTQK SLSLSPGAGG GGSGGGGSGG GGSITCPPPM SVEHADIWVK
SYSLYSRERY ICNSGFKRKA GTSSLTECVL NKATNVAHWT TPSLKCIRGG GGSGGGSGGG
GSAAGGGGSG GGGSGGGGSN WVNVISDLKK IEDLIQSMHI DATLYTESDV HPSCKVTAMK
CFLLELQVIS LESGDASIHD TVEDLIILAN NSLSSNGNVT ESGCKECEEL EEKNIKEFLQ
SFVHIVQMFI NTS
SEQ ID NO: 53 - CTLA-4 antibody Ipilimumab HC- IL-21
QVQLVESGGG VVQPGRSLR LSCAASGFTF SSYTMHWVRQ APGKGLEWVT FISYDGNNKYY
ADSVKGRFTI SRDNSKNTL YLQMNSLRAE DTAIYYCART GWLGPFDYWG QGTLVTVSSAS
TKGPSVFPLA PSSKSTSGG TAALGCLVKD YFPEPVTVSW NSGALTSGVH TFPAVLQSSGL
YSLSSVVTVP SSSLGTQTY ICNVNHKPSN TKVDKRVEPK SCDKTHTCPP CPAPELLGGPS
VFLFPPKPKD TLMISRTPE VTCVVVDVSH EDPEVKFNWY VDGVEVHNAK TKPREEQYNST
YRVVSVLTVL HQDWLNGKE YKCKVSNKAL PAPIEKTISK AKGQPREPQV YTLPPSRDELT
KNQVSLTCLV KGFYPSDIA VEWESNGQPE NNYKTTPPVL DSDGSFFLYS KLTVDKSRWQQ
GNVFSCSVMH EALHNHYTQ KSLSLSPGA (GGGGS)ₙ₁X₁X₂(GGGGS)ₙ₂QGQDRHMIRM RQLI
DIVDQLKNYV NDLVPEFLP APEDVETNCE WSAFSCFQKA QLKSANTGNN ERIINVSIKKL
KRKPPSTNAG RRQKHRLTC PSCDSYEKKP PKEFLERFKS LLQKMIHQHL SSRTHGSEDS
Wherein n₁ = 0, 1, 2, or 3; n₂ = 0, 1, 2, or 3; X₁ is an amino acid
Alanine or deleted; and X₂ is an amino acid Alanine or deleted.
SEQ ID NO: 54 - CTLA-4 antibody Ipilimumab HC-IL-2Rβ-ECD
QVQLVESGGG VVQPGRSLRL SCAASGFTFS SYTMHWVRQA PGKGLEWVTF ISYDGNNKYY
ADSVKGRFTI SRDNSKNTLY LQMNSLRAED TAIYYCARTG WLGPFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSRDELT
KNQVSLTCLV KGFYPSDIAV EWESNGQPEN NYKTTPPVLD SDGSFFLYSK LTVDKSRWQQ
GNVFSCSVMH EALHNHYTQK SLSLS PGA (GGGGS)ₙ₁X₁X₂(GGGGS)ₙ₂ AVNG TSQFTCFYNS
RANISCVWSQ DGALQDTSCQ VHAWPDRRRW NQTCELLPVS QASWACNLIL GAPDSQKLTT
VDIVTLRVLC REGVRWRVMA IQDFKPFENL RLMAPISLQV VHVETHRCNI SWEISQASHY
FERHLEFEAR TLSPGHTWEE APLLTLKQKQ EWICLETLTP DTQYEFQVRV KPLQGEFTTW
SPWSQPLAFR TKPAALGKDT
Wherein n₁ = 0, 1, 2, or 3; n₂ = 0, 1, 2, or 3;
X₁ is an amino acid Alanine or deleted;
and X₂ is an amino acid Alanine or deleted.
SEQ ID NO: 55 - CTLA-4 antibody Ipilimumab HC-scFv1 against IL-2 (in
the scFV, underlined: HCDR1-3 and LCDR1-3; italic: VH and VL)
QVQLVESGGG VVQPGRSLRL SCAASGFTFS SYTMHWVRQA PGKGLEWVTF ISYDGNNKYY
ADSVKGRFTI SRDNSKNTLY LQMNSLRAED TAIYYCARTG WLGPFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSRDELT
KNQVSLTCLV KGFYPSDIAV EWESNGQPEN NYKTTPPVLD SDGSFFLYSK LTVDKSRWQQ
GNVFSCSVMH EALHNHYTQK SLSLSPGA(GGGGS)ₙ₁X₁X₂(GGGGS)ₙ₂QVQ LVQSGAEVEE
PGSSVKVSCK ASGGTFSSYA ISWVRQAPGQ GLEWMGGIIP IFGTANYAQK LVQSGAEVKK
ESTSSAYMEL SSLRSEDTAV YYCARVDRYY NWNYFLGSFD YWGQGTLVTV SSGGGGSGGG
GSGGGGSSYV LTQPPSVSVA PGKTARITCG GNNIRSKSVH WYQQKPGQAP VVVIYYDSDR
PSGIPERISG SBSGNTATLITSRVEAGDEADYFCQVWDSSSDHHVFGGGTKLTVL;
Wherein n₁ = 0, 1, 2, or 3; n₂ = 0, 1, 2, or 3; X₁ is an amino acid
Alanine or deleted; and X₂ is an amino acid Alanine or deleted.
SEQ ID NO: 56 - CTLA-4 antibody Ipilimumab HC- scFv2 against IL-2
(underlined: LCDR1-3 and HCDR1-3; italic: VL and VH)
QVQLVESGGG VVQPGRSLRL SCAASGFTFS SYTMHWVRQA PGKGLEWVTF ISYDGNNKYY
ADSVKGRFTI SRDNSKNTLY LQMNSLRAED TAIYYCARTG WLGPFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSRDELT
KNQVSLTCLV KGFYPSDIAV EWESNGQPEN NYKTTPPVLD SDGSFFLYSK LTVDKSRWQQ
GNVFSCSVMH EALHNHYTQK SLSLSPGA (GGGGS)ₙ₁X₁X₂ (GGGGS)ₙ₂SYVL TQPPSVSVAP
GKTARITCGG NNIRSKSVHW YQQKPGQAPV VVIYYDSDRP SGIPERISGS NSGNTATLTI
SRVEAGDEAD YFCQVWDSSS DHHVFGGGTK LTVLGGGGSG GGGSGGGGSQ VQLVQSGAEV
KKPGSSVKVS CKASGGTFSS YAISWVRQAP GQGLEWMGGI IPIFGTANYA QKFQGRVTIT
ADESTSTAYM ELSSLRSEDT AVYYCARVDR YYNWNYFLGS FDYWGQGTLV TVSS;
Wherein n₁ = 0, 1, 2, or 3; n₂ = 0, 1, 2, or 3; X₁ is an amino acid
Alanine or deleted; and X₂ is an amino acid Alanine or deleted.
SEQ ID NO: 57 - CTLA-4 antibody Ipilimumab HC- scFv2 against IL-15
QVQLVESGGG VVQPGRSLRL SCAASGFTFS SYTMHWVRQA PGKGLEWVTF ISYDGNNKYY
ADSVKGRFTI SRDNSKNTLY LQMNSLRAED TAIYYCARTG WLGPFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSRDELT
KNQVSLTCLV KGFYPSDIAV EWESNGQPEN NYKTTPPVLD SDGSFFLYSK LTVDKSRWQQ
GNVFSCSVMH EALHNHYTQK SLSLSPGA (GGGGS)ₙ₁X₁X₂ (GGGGS)n2EIVL TQSPGTLSLS
PGERATLSCR ASQSVSSSYL AWYQQKPGQA PRLLIYGASR RATGIPDRFS GSGSGTDFTL
TISRLEPEDF AVYYCQRYGS SHTFGQGTKL EISGGGGSGG GGSGGGGSEV QLVQSGAEVK
KPGESLKISC KVSGYFFTTY WIGWVRQMPG KGLEYMGIIY PGDSDTRYSP SFQGQVTISA
DKSISTAYLQ WSSLKASDTA MYYCARGGNW NCFDYWGQGT LVTVSS
Wherein n₁ = 0, 1, 2, or 3; n₂ = 0, 1, 2, or 3; X₁ is an amino acid
Alanine or deleted; and X₂ is an amino acid Alanine or deleted.
SEQ ID NO: 58 - CTLA-4 antibody Ipilimumab HC- IL-21R-ECD
QVQLVESGGG VVQPGRSLRL SCAASGFTFS SYTMHWVRQA PGKGLEWVTF ISYDGNNKYY
ADSVKGRFTI SRDNSKNTLY LQMNSLRAED TAIYYCARTG WLGPFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSRDELT
KNQVSLTCLV KGFYPSDIAV EWESNGQPEN NYKTTPPVLD SDGSFFLYSK LTVDKSRWQQ
GNVFSCSVMH EALHNHYTQK SLSLSPGAGG GGSGGGGSAA GGGGSGGGGS CPDLVCYTDY
LQTVICILEM WNLHPSTLTL TWQDQYEELK DEATSCSLHR SAHNATHATY TCHMDVFHFM
ADDIFSVNIT DQSGNYSQEC GSFLLAESIK PAPPFNVTVT FSGQYNISWR SDYEDPAFYM
LKGKLQYELQ YRNRGDPWAV SPRRKLISVD SRSVSLLPLE FRKDSSYELQ VRAGPMPGSS
YQGTWSEWSD PVIFQTQSEE LKE
SEQ ID NO: 59 - CTLA-4 antibody Ipilimumab HC- scFv2 against IL-21
QVQLVESGGG VVQPGRSLRL SCAASGFTFS SYTMHWVRQA PGKGLEWVTF ISYDGNNKYY
ADSVKGRFTI SRDNSKNTLY LQMNSLRAED TAIYYCARTG WLGPFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSRDELT
KNQVSLTCLV KGFYPSDIAV EWESNGQPEN NYKTTPPVLD SDGSFFLYSK LTVDKSRWQQ
GNVFSCSVMH EALHNHYTQK SLSLSPGAGG GGSGGGGSAA GGGGSGGGGS EIVLTQSPGT
LSLSPGERAT LSCRASQSVS SSYLAWYQQK PGQAPRLLIY GASSRATGIP DRFSGSGSGT
DFTLTISRLE PEDFAVYYCQ QYGSWTFGQG TKVEIKGGGG SGGGGSGGGG SQVQLVESGG
GVVQPGRSLR LSCAASGFTF SSYGMHWVRQ APGKGLEWVA FIWYDGSDKY YADSVKGRFT
ISRDNSKNTL YLQMNSLRAE DTAVYYCARD GDSSDWYGDY YFGMDVW-GQGTTVTVSS
SEQ ID NO: 60 - 4E12B2D10 anti-IL2 HC variable domain (underlined:
HCDR1-3)
EVQLQQSGAE LVRPGASVKL SCTASGFNIK DDYLHWVRQR PEQGLEWIGR IDPANGNTKY
APKFQDKATI TADTSSNTAY LQLSILTSED TAVYYCAARF GYFYGSSFYA VAYWGQGTSV
TVSS
SEQ ID NO: 61 - 4E12B2D10 anti-IL2 LC variable domain (underlined:
LCDR1-3)
QIVLTQSPAI MSASPGEKVT MTCSASSSVS YMHWYQQKSG TSPKRWIYDT SKLASGVPAR
FSGSGSGTSY SLTISSMEAE DAATYYCQQW SSNPYTFGGGTKLEIK
SEQ ID NO: 62 - IL-2 agonist polypeptide
APXₐₐ₃SSSTK KTQLQLEHLL LDLQMILNGI NNYKNPXₐₐ₃₅LTRMLTXₐₐ₄ZKFXₐₐ₄sMPKKATEL
KHLQCLEEEL KPLEEVLNXₐₐ₇₂Xₐₐ₇₃QSKNFH LRPRDLISNI NVIVLELKGSE TTFMCEYADE
TATIVEFLNR WITFXₐₐ₁₂₅QS IISTLT
wherein Xₐₐ₃ is N or A; wherein Xₐₐ₁₂₅ is C, A or S; wherein Xₐₐ₃₅ is
selected from K and N; wherein Xₐₐ₄₂ is selected from A, G, S, T, Q,
E, N, D, R, and K; wherein Xₐₐ₄₅ is selected from A, G, S, T, Q, E, N,
D, R, and K; wherein Xₐₐ₇₂ is selected from A, G, S, T, Q, E, N, D, R,
and K; and wherein Xₐₐ₇₃ is selected from A and T.
SEQ ID NO: 63 - IL-21 receptor alpha ECD mutant ver1 (mutations
underlined)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SDYEDPAFYM LKGKLQYELQ YRNRGDGSGV SPRRKLISVD SRSVSLLPLE FRKDSSYELQ
VRAGPMPGSS YQGTWSEWSD PVIFQTQSEE LKE
SEQ ID NO: 64 - IL-21 receptor alpha ECD mutant ver2 (mutations
underlined)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SDYEDPAFYM LKGKLQYELQ YRNRGDNASV SPRRKLISVD SRSVSLLPLE FRKDSSYELQ
VRAGPMPGSS YQGTWSEWSD PVIFQTQSEE LKE
SEQ ID NO: 65 - IL-21 receptor alpha ECD mutant ver3 (mutations
underlined)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SDYEDPAFYM LKGKLQYELQ YRNRGDNGSV SPRRKLISVD SRSVSLLPLE FRKDSSYELQ
VRAGPMPGSS YQGTWSEWSD PVIFQTQSEE LKE
SEQ ID NO: 66 - TL-21 receptor alpha ECD mutant ver4 (mutations
underlined)
LQTVICILEM WNLHPSTLTL TWQDQYEELK DEATSCSLNR SAHNATHATY CPDLVCYTDY
TCHMDVFHFM ADDIFSVNIT DQSGNYSQEC GSFLLAESIK PAPPFNVTVT FSGQYNISWR
SDYEDPAFYM LKGKLQYELQ YRNRGDNASV SPRRKLISVD SRSVSLLPLE FRKDSSYELQ
VRAGPMPGSS YQGTWSEWSD PVIFQTQSEE LKE
SEQ ID NO: 67 - IL-21 receptor alpha ECD mutant ver5 (mutations
underlined)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SDYEDPAFYM LKGKLQYELQ YRNRGDPSAV SPRRKLISVD SRSVSLLPLE FRKDSSYELQ
VRAGPMPGSS YQGTWSEWSD PVIFQTQSEE LKE
SEQ ID NO: 68 - IL-21 receptor alpha ECD mutant ver6 (mutations
underlined)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SDYEDPAFYM LKGKLQYELQ YRNRGDPSGV SPRRKLISVD SRSVSLLPLE FRKDSSYELQ
VRAGPMPGSS YQGTWSEWSD PVIFQTQSEE LKE
SEQ ID NO: 69 - IL-21 receptor alpha ECD mutant ver7 (mutations
underlined)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SAYEDPAFYM LKGKLQYELQ YRNRGDGSGV SPRRKLISVD SRSVSLLPLE FRKDSSYELQ
VRAGPMPGSS YQGTWSEWSD PVIFQTQSEE LKE
SEQ ID NO: 70 - IL-21 receptor alpha ECD mutant ver8 (mutations
underlined)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SIYEDPAFYM LKGKLQYELQ YRNRGDGSGV SPRRKLISVD SRSVSLLPLE FRKDSSYELQ
VRAGPMPGSS YQGTWSEWSD PVIFQTQSEE LKE
SEQ ID NO: 71 - IL-21 receptor alpha ECD mutant ver9 (mutations
underlined)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SWYEDPAFYM LKGKLQYELQ YRNRGDGSGV SPRRKLISVD SRSVSLLPLE FRKDSSYELQ
VRAGPMPGSS YQGTWSEWSD PVIFQTQSEE LKE
SEQ ID NO: 72 - IL-21 receptor alpha ECD mutant ver10 (mutations
underlined)
CPDLVCYTDY LQTVICILEM WNLHPSTLTL TWQDQYEELK DEATSCSLHR SAHNATHAT
TCHMDVFHFM ADDIFSVNIT DQSGNYSQEC GSFLLAESIK PAPPFNVTVT FSGQYNISWR
SFYEDPAFYM LKGKLQYELQ YRNRGDGSGV SPRRKLISVD SRSVSLLPLE FRKDSSYELQ
VRAGPMPGSS YQGTWSEWSD PVIFQTQSEE LKE
SEQ ID NO: 73 - IL-21 receptor alpha ECD mutant ver11
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SYYEDPAFYM LKGKLQYELQ YRNRGDGSGV SPRRKLISVD SRSVSLLPLE FRKDSSYELQ
VRAGPMPGSS YQGTWSEWSD PVIFQTQSEE LKE
SEQ ID NO: 74 - PD1-HC with hole mutations IL-21Rα ECD
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VCTLPPSQEE MTKNQVSLSC
AVKGFYPSDI AVEWESNGQP ENNYKTTPPV LDSDGSFFLV SRLTVDKSRW QEGNVFSCSV
MHEALHNHYT QKSLSLSLGA GGGGSGGGGS GPLGVRGGGG SCPDLVCYTD YLQTVICILE
MWNLHPSTLT LTWQDQYEEL KDEATSCSLH RSAHNATHAT YTCHMDVFHF MADDIFSVNI
TDQSGNYSQE CGSFLLAESI KPAPPFNVTV TFSGQYNISW RSDYEDPAFY MLKGKLQYEL
QYRNRGDPWA VSPRRKLISV DSRSVSLLPL EFRKDSSYEL QVRAGPMPGS SYQGTWSEWS
DPVIFQTQSE ELKE
SEQ ID NO: 75 - PD1-HC fusion polypeptide with scFv2 against IL-15
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGA GGGGSGGGGS GPLGVRGGGG SGGGGSEIVL TQSPGTLSLS
PGERATLSCR ASQSVSSSYL AWYQQKPGQA PRLLIYGASR RATGIPDRFS GSGSGTDFTL
TISRLEPEDF AVYYCQRYGS SHTFGQGTKL EISGGGGSGG GGSGGGGSEV QLVQSGAEVK
KPGESLKISC KVSGYFFTTY WIGWVRQMPG KGLEYMGIIY PGDSDTRYSP SFQGQVTISA
DKSISTAYLQ WSSLKASDTA MYYCARGGNW NCFDYWGQGT LVTVSS
SEQ ID NO: 76 - Anti-IL-15 antibody 146B7 HC CDR1 (protein sequence)
TYWIG
SEQ ID NO: 77 - Anti-IL-15 antibody 146B7 HC CDR2 (protein sequence)
IIYPGDSDTR YSPSFQG
SEQ ID NO: 78 - Anti-IL-15 antibody 146B7 HC CDR3 (protein sequence)
GNWNCFDY
SEQ ID NO: 79 - Anti-IL-15 antibody 146B7 LC CDR1 (protein sequence)
RASQSVSSSYLA
SEQ ID NO: 80 - Anti-IL-15 antibody 146B7 LC CDR2 (protein sequence)
GASRRAT
SEQ ID NO: 81 - Anti-IL-15 antibody 146B7 LC CDR3 (protein sequence)
QRYGSSHT
SEQ ID NO: 82 - Anti-IL-15 antibody 146B7 HC CDR3 ver2 (protein
sequence)
GNWNSFDY
SEQ ID NO: 83 - Anti-IL-15 antibody 146B7 HC variable domain
(underlined: HCDR1-3)
EVQLVQSGAE VKKPGESLKI SCKVSGYFFT TYWIGWVRQM PGKGLEYMGI IYPGDSDTRY
SPSFQGQVTI SADKSISTAY LQWSSLKASD TAMYYCARGG NWNCFDYWGQ GTLVTVSS
SEQ ID NO: 84 - Anti-IL-15 antibody 146B7 LC variable domain
(underlined: LCDR1-3)
EIVLTQSPGT LSLSPGREAT LSCRASQSVS SSYLAWYQQK PGQAPRLLIY GASRRATGIP
DRFSGSGSGT DFTLTISRLE PEDFAVYYCQ RYGSSHTFGQ GTKLEISRTV AAPSVFIFP

1. A prodrug comprising a cytokine moiety, a masking moiety, and a carrier moiety, wherein:
a) the masking moiety binds to the cytokine moiety and inhibits a biological activity of the cytokine;
b) the carrier moiety comprises an antigen-binding moiety, wherein the antigen-binding moiety binds to an antigen expressed on the surface of a target cell; and
c) the masking moiety is linked directly or indirectly to the carrier moiety through a non-cleavable peptide linker or without a peptide linker.

2. The prodrug of claim 1, wherein the prodrug is activated by cells that express on the cell surface both the antigen and a receptor for the cytokine moiety, optionally wherein the biological activity of the prodrug is increased by at least 2, 5, or 10 folds at a disease site comprising the cells as compared to a site not comprising the cells.

3. The prodrug of claim 1 or 2, wherein the receptor for the cytokine moiety comprises two or more subunits.

4. The prodrug of any one of the preceding claims, wherein the antigen is selected from PD-1, PD-L1, CTLA-4, TIGIT, TIM-3, LAG-3, CD25, CD16a, CD16b, NKG2D, NKP44, NKP30, CD19, CD20, CD30, CD38, BCMA, and signal regulatory protein alpha (SIRP alpha).

5. The prodrug of any one of the preceding claims, wherein the cytokine moiety comprises a an IL-2 agonist polypeptide, an IL-7 agonist polypeptide, an IL-9 agonist polypeptide, an IL-15 agonist polypeptide, an IL-21 agonist polypeptide, an IL-1α agonist polypeptide, an IL-1β agonist polypeptide, an IL-4 agonist polypeptide, an IL-5 agonist polypeptide, an IL-6 agonist polypeptide, an IL-8 agonist polypeptide, an IL-10 agonist polypeptide, an IL-12 agonist polypeptide, an IL-17 agonist polypeptide, an IL-18 agonist polypeptide, an IL-22 agonist polypeptide, an IL-23 agonist polypeptide, an IL-31 agonist polypeptide, an IL-33 agonist polypeptide, an IL-36 agonist polypeptide, an interferon-alpha agonist polypeptide, an interferon gamma agonist polypeptide, a 4-1BB ligand, an OX-40 ligand, or a CD-40 ligand.

6. The prodrug of any one of the preceding claims, wherein the masking moiety comprises an ECD of a receptor of the cytokine, or an antigen-binding domain that specifically binds the cytokine.

7. The prodrug of any one of the preceding claims, wherein
i) the cytokine moiety comprises an IL-7 agonist polypeptide and the masking moiety comprises an extracellular domain (ECD) of IL-7 receptor α (IL-7Rα ECD) or a functional analog thereof,
ii) the cytokine moiety comprises an IL-21 agonist polypeptide and the masking moiety comprises an ECD of IL-21 receptor α (IL-21Rα ECD) or a functional analog thereof,
iii) the cytokine moiety comprises an IL-2 agonist polypeptide or an IL-15 agonist polypeptide and the masking moiety comprises an ECD of IL-2 receptor β (IL-2Rβ ECD) or a functional analog thereof,
iv) the cytokine moiety comprises an IL-21 agonist polypeptide and the masking moiety comprises a Fab, a single chain Fv (scFv), or a single domain antibody against IL-21,
v) the cytokine moiety comprise an IL-2 agonist polypeptide and the masking moiety comprises a Fab, an scFv, or a single domain antibody against IL-2,
vi) the cytokine moiety comprises an IL-15 agonist polypeptide and the masking moiety comprises a Fab, an scFv, or a single domain antibody against IL-15, or
vii) the cytokine moiety comprises an IL-15 agonist polypeptide and the masking moiety comprises a sushi domain of IL-15 receptor α (IL-15Rα sushi domain).

8. The prodrug of any one of claims 5-7, wherein the cytokine moiety comprises an IL-2 agonist polypeptide comprising SEQ ID NO: 6 or 62, or an amino acid sequence at least 95% identical thereto.

9. The prodrug of any one of claims 5-7, wherein the cytokine moiety comprises an IL-15 agonist polypeptide comprising SEQ ID NO: 7, or an amino acid sequence at least 95% identical thereto.

10. The prodrug of any one of claims 5-9, wherein the cytokine moiety comprises an IL-2 or IL-15 agonist polypeptide and the masking moiety comprises an IL-2Rβ ECD or a functional analog thereof, optionally wherein the IL-2-Rβ ECD comprises SEQ ID NO: 11, or an amino acid sequence at least 95% identical thereto.

11. The prodrug of any one of claims 5-9, wherein the cytokine moiety comprises an IL-2 agonist polypeptide and the masking moiety comprises an scFv against IL-2 and optionally inhibits or interferes with the interaction between the IL-2 cytokine moiety and one or more of IL-2Rα, IL-2Rβ, and IL-2Rγ, optionally wherein the scFv comprises
the HCDR1-3 and LCDR1-3, or the heavy and light chain variable domains, of antibody 4E12B2D10, Anti-IL-2 scFv1, or Anti-IL-2 scFv2, or
an amino acid sequence comprising SEQ ID NO: 22, SEQ ID NO: 23, or SEQ ID NOs: 60 and 61, or an amino acid sequence at least 95% identical thereto.

12. The prodrug of any one of any one of claims 5-9, wherein the cytokine moiety comprises an IL-15 agonist polypeptide and the masking moiety comprises an scFv against IL-15 and optionally inhibits or interferes with the interaction between the IL-15 cytokine moiety and one or more of IL-15Rα, IL-2Rβ, and IL-2Rγ, optionally wherein the scFv comprises
the HCDR1-3 and LCDR1-3, or the heavy and light chain variable domains, of antibody 146B7, Anti-IL-15 scFv1, or Anti-IL-15 scFv2, or
an amino acid sequence comprising SEQ ID NO: 18, SEQ ID NO: 19, or SEQ ID NOs: 83 and 84, or an amino acid sequence at least 95% identical thereto.

13. The prodrug of any one of claims 5-7, wherein the cytokine moiety comprises an IL-21 agonist polypeptide comprising any one of SEQ ID NOs: 1-5, or an amino acid sequence at least 95% identical thereto.

14. The prodrug of any one of claims 5-7 or 13, wherein the cytokine moiety comprises an IL-21 agonist polypeptide and the masking moiety comprises an IL-21Rα ECD or a functional analog thereof, or an IL-21Rγ ECD or a functional analog thereof, optionally wherein the masking moiety comprises SEQ ID NO: 12, 13, or any one of SEQ ID NOs: 63-73, or an amino acid sequence at least 95% identical thereto.

15. The prodrug of any one of claims 5-7 or 13, wherein the cytokine moiety comprises an IL-21 agonist polypeptide and the masking moiety comprises an scFv against human IL-21 and optionally inhibits or interferes with the interaction between the IL-21 cytokine moiety and IL-21Rα and/or IL-21Rγ, optionally wherein the scFv comprises
the HCDR1-3 and LCDR1-3, or the heavy and light chain variable domains, of Anti-IL-21 scFv1, or Anti-IL-21 scFv2, or
an amino acid sequence comprising SEQ ID NO: 20 or 21, or an amino acid sequence at least 95% identical thereto.

16. The prodrug of any one of the preceding claims, wherein the masking moiety comprises
an scFv against a cytokine selected from IL-2, IL-4, IL-7, IL-9, IL-15, and IL-21 and optionally inhibits or interferes with the interaction between the cytokine and IL-2Rγ, or
an ECD of a receptor α or γ of a cytokine selected from IL-2, IL-4, IL-7, IL-9, IL-15, and IL-21.

17. The prodrug of any one of the preceding claims, wherein the prodrug comprises an anti-PD-1 antibody moiety, wherein the antibody moiety comprises two light chains comprising SEQ ID NO: 44, and two heavy chains comprising respectively:
SEQ ID NOs: 24 and 25,
SEQ ID NOs: 24 and 26 or 74,
SEQ ID NOs: 35 and 36,
SEQ ID NOs: 37 and 36,
SEQ ID NOs: 37 and 38 or 75,
SEQ ID NOs: 39 and 41 or 74, or
SEQ ID NOs: 42 and 43 or 74.

18. The prodrug of any one of the preceding claims, wherein the prodrug comprises an anti-CTLA4 antibody moiety, wherein the antibody moiety comprises two light chains comprising SEQ ID NO: 50, and two heavy chains comprising respectively:
SEQ ID NOs: 51 and 54,
SEQ ID NOs: 51 and 55,
SEQ ID NOs: 51 and 56,
SEQ ID NOs: 52 and 54,
SEQ ID NOs: 53 and 58,
SEQ ID NOs: 53 and 59, or
SEQ ID NOs: 52 and 57.

19. A pharmaceutical composition comprising the prodrug of any one of claims 1-18 and a pharmaceutically acceptable excipient.

20. A polynucleotide or polynucleotides encoding the prodrug of any one of claims 1-18.

21. An expression vector or vectors comprising the polynucleotide or polynucleotides of claim 20.

22. A host cell comprising the vector(s) of claim 21, optionally wherein the host cell is a mammalian cell.

23. A method of making the prodrug of any one of claims 1-18, comprising
a) culturing the host cell of claim 22 under conditions that allow expression of the prodrug wherein the host cell is a mammalian cell; and
b) isolating the prodrug from the cell culture.

24. A method of treating a cancer, an autoimmune disease, or an infectious disease, or stimulating the immune system in a patient in need thereof, comprising administering to the patient a therapeutically effective amount of a prodrug of any one of claims 1-18 or the pharmaceutical composition of claim 19.

25. A prodrug of any one of claims 1-18 or the pharmaceutical composition of claim 19 for use in treating a cancer, an autoimmune disease or an infectious disease, or stimulating the immune system, in a patient in need thereof.

26. Use of a prodrug of any one of claims 1-18 for the manufacture of a medicament for treating a cancer, an autoimmune disease, or an infectious disease, or stimulating the immune system, in a patient in need thereof.

27. The method of claim 24, the prodrug or pharmaceutical composition for use of claim 25, or the use of claim 26, wherein the patient has
an HIV, HBV, HCV, or HPV infection,
an autoimmune disease selected from lupus, type I diabetes, psoriasis, dermatomyositis, GvHD, or rheumatoid arthritis, or
a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, or stomach cancer.
